(19) United States
(12) Patent Application Publication
(10) Pub. No.: US 2005/0004218 A1
(43) Pub. Date: Jan. 6, 2005

(54) Vaccenic acid

(76) Inventors: Dale E. Bauman, Ithaca, NY (US); Clement Ip, Orchard Park, NY (US); Benjamin A. Corl, Raleigh, NC (US)

Correspondence Address: Schwegman, Lundberg, Woessner & Kluth, P.A., P.O. Box 2938, Minneapolis, MN, 55402, US

(21) Appl. No.: 10/839,808

(22) Filed: May 5, 2004

Related U.S. Application Data
(60) Provisional application No. 60/467,897, filed on May 5, 2003.

(51) Int. Cl. A61K 31/202

(52) U.S. Cl. 514/560

(57) ABSTRACT

The invention relates to compositions and methods involving vaccenic acid. Such compositions and methods are useful for preventing cancer.

The invention described herein was made with United States Government support under Grant Numbers CA 61763 and CA 16056 awarded by the National Cancer Institute. The United States Government has certain rights in this invention.

### FIELD OF THE INVENTION

The invention relates to compositions and methods employing vaccenic acid (trans-11 18:1 octadecenoic acid). Such compositions and methods are useful for preventing and treating cancer, for example, breast cancer.

### BACKGROUND OF THE INVENTION

Chemotherapy typically involves the use of drugs that aim either to kill cancer cells or to stop the spread of cancer. Chemotherapy may be used either alone or in combination with one or more invasive or destructive forms of cancer treatment, including surgery or radiotherapy. Most chemotherapy regimes, other than those primarily targeted at the endocrine system such as anti-oestrogens and antiandrogens, cause significant side effects. The side effects include nausea, vomiting, suppression of the immune system, suppression of white blood cells and platelets, hair loss, cardiovascular damage, lung damage, renal damage, nerve damage and marked fatigue and malaise. These side effects differ from drug to drug, but it is now common to use two, three, four or more drugs in combination in chemotherapy regimes so that most chemotherapy-treated patients will experience one or more side effects. Each drug has a specific range of side effects, some of which may be particularly significant and limit the dose of the drug that can be given. Doxorubicin and related compounds, for example, can be severely cardiotoxic and this is a common dose-limiting side effect. Bleomycin, and to a lesser extent cyclophosphamide, can be toxic to the lungs causing fibrosis. The platinum derivatives and related compounds may be very toxic to the nerves.

The term “functional foods” has been adopted to illustrate the concept that microcomponents of foods can provide health benefits beyond those associated with traditional nutrients. A few substances in our diet have been identified as anticarcinogens based on biomedical studies with animal models. The intake of these anticarcinogens may therefore aid in the prevention of cancer. However, only a few anti-cancer functional foods have been identified.

Accordingly, new, functional foods that have anti-cancer properties are needed. Similarly, less toxic methods are needed for preventing and treating cancer.

### SUMMARY OF THE INVENTION

According to the invention, vaccenic acid (trans-11 octadecenoic acid) is a potent anti-cancer agent.

Hence, the invention provides a composition comprising vaccenic acid and a carrier. In some embodiments, administration of the composition comprising vaccenic acid can decrease tumor formation in mammary tissues.

Such compositions can have a therapeutically effective amount or a preventative amount of vaccenic acid. A therapeutically effective amount can, for example, be sufficient to decrease tumor formation in a mammal. A preventative amount of vaccenic acid can prevent the formation of cancers, for example, tumors in a mammal. In some embodiments, an effective amount can increase tissue accumulation of vaccenic acid or conjugated linoleic acid in a mammal. In other embodiments, an effective amount can increase accumulation of vaccenic acid or conjugated linoleic acids in mammary tissues. Such a preventative or therapeutically effective amount can, for example, be between about 0.5 mg and about 50 gm, or between about 250 mg and about 5 gm.

The composition can comprise a triacylglycerol of vaccenic acid.

The composition can be a food product. Such a food product can be a diet drink, diet bar, cookie, health bar, salad dressing, sauce, cream, whipped cream, ice cream, butter, milk shake, health drink, powdered drink mix, powdered cake mix, powdered snack bar mix, margarine, cooking oil, lard, prepared frozen meal, candy, cracker, snack chip, meat product, milk, cheese, or yogurt.

The composition can also be a health supplement, for example, a tablet or capsule.

The invention is also directed to a method for preventing or treating cancer in a mammal comprising administering vaccenic acid to the mammal. For example, the cancer can be melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate carcinoma, adenocarcinoma, ovarian adenocarcinoma, renal adenocarcinoma, mammary adenocarcinoma, lung adenocarcinoma, pancreatic adenocarcinoma or non-seminomal testis carcinoma tissues. In some embodiments, the cancer is breast cancer.

The invention also provides a method for increasing tissue accumulation of conjugated linoleic, for example, cis-9, trans-11 conjugated linoleic acid (CLA), in a mammal. The method involves administering vaccenic acid to the mammal. The National Academy of Sciences publication, Carcinogens and Anticarcinogens in the Human Diet, concluded “conjugated linoleic acid (CLA) is the only fatty acid shown unequivocally to inhibit carcinogenesis in experimental animals” (National Research Council/National Academy of Sciences, 1996). In the present invention, the inventors have for the first time identified vaccenic acid as a second fatty acid that is an effective anticarcinogen.

### DESCRIPTION OF THE FIGURES

FIG. 1A-C illustrate that the liver (FIG. 1A), plasma (FIG. 1B), and mammary fat pad (FIG. 1C) content of vaccenic acid (VA) increases dramatically as increasing amounts of vaccenic acid are provided in the diet. However, increased dietary content of cis-9, trans-11 conjugated linoleic acid did not significantly affect vaccenic acid accumulation in tissues. The amount of cis-9, trans-11 conjugated linoleic acid provided in the diet is shown along the x-axis and the amount of vaccenic acid in the tissues is shown on the y-axis. Diets A, B, C, and D contained an identical concentration of vaccenic acid (0.13%) and increasing concentrations of cis-9, trans-11 conjugated linoleic acid (0.05, 0.18, 0.24 and 0.37%, respectively). Diets E, F, and G contained cis-9, trans-11 conjugated linoleic acid that matched diets B, C, and D, respectively, and increasing concentrations of vaccenic acid (0.73, 1.00, and 1.60%, respectively). As shown, the lower portion of the bars that indicate the amount of vaccenic acid in tissues of animals fed diets A-D, is lower than the upper bars that indicate the amount of vaccenic acid in tissues of animals fed diets E-G.

FIG. 2A-C illustrate that the liver (FIG. 2A), plasma (FIG. 2B), and mammary fat pad (FIG. 2C) content of cis-9, trans-11 conjugated linoleic acid increases dramatically as increasing amounts of vaccenic acid are provided in the diet. The dietary content of cis-9, trans-11 conjugated linoleic acid is shown along the x-axis. Diets A, B, C, and D contained an identical concentration of vaccenic acid (0.13%) and increasing concentrations of cis-9, trans-11 conjugated linoleic acid (0.05, 0.18, 0.24 and 0.37%, respectively). Diets E, F, and G contained cis-9, trans-11 conjugated linoleic acid that matched diets B, C, and D, respectively, and increasing concentrations of vaccenic acid (0.73, 1.00, and 1.60%, respectively). Standard errors of the mean ranged from 0.015 to 0.084, 0.014 to 0.090, and 0.016 to 0.128 for liver, plasma, and mammary fat pad content of cis-9, trans-11 conjugated linoleic acid, respectively. As shown, the lower portion of the bars that indicate the amount of cis-9, trans-11 CLA in tissues of animals fed diets A-D, is lower than the upper bars that indicate the amount of cis-9, trans-11 CLA in tissues of animals fed diets E-G. The increase in CLA content is related to the fact that vaccenic acid can be converted to cis-9, trans-11 conjugated linoleic acid (CLA) by the enzyme Δ⁹-desaturase. Δ⁹-desaturase, also called stearoyl CoA desaturase, is an enzyme present in many mammalian tissues including liver, adipose tissue and mammary tissue.

FIG. 3 illustrates the relationship between tissue content of vaccenic acid and cis-9, trans-11 conjugated linoleic acid (CLA) in liver, plasma, and mammary fat pad of rats fed varying concentrations of vaccenic acid (trans-11 18:1) and cis-9, trans-11 conjugated linoleic acid. Data points represent CLA and vaccenic acid concentrations of individual animals for liver (closed square), plasma (open triangle), and mammary fat pad (closed circle) tissues. For reference, an insert is provided showing the relationship between vaccenic acid and cis-9, trans-11 conjugated linoleic acid in the diet (open diamond), where x-axis and y-axis units are the same as in the larger graph. The differences among tissues and plasma in the slope of this line are a reflection of the role of Δ⁹-desaturase in converting vaccenic acid to cis-9, trans-11 CLA.

FIG. 4 graphically illustrates the relationship between plasma content of vaccenic acid (VA) and mammary fat pad content of vaccenic acid from rats fed varying concentrations of vaccenic acid (trans-11 18:1) and cis-9, trans-11 conjugated linoleic acid.

FIG. 5 graphically illustrates the relationship between plasma content of cis-9, trans-11 conjugated linoleic acid and mammary fat pad content of cis-9, trans-11 conjugated linoleic acid from rats fed varying concentrations of vaccenic acid (trans-11 18:1) and cis-9, trans-11 conjugated linoleic acid.

FIGS. 6A-6D graphically illustrate the effect of sterculic oil, an inhibitor of the Δ⁹-desaturase enzyme system, on fatty acid precursors and products of the Δ⁹-desaturase enzyme system in the mammary fat pad for various diets (A-D). Diet regimens A-D are described in Table 12. FIG. 6A provides the g/100 g fatty acids of 14:0 and cis-9 14:1 fatty acids. FIG. 6B provides the g/100 g fatty acids of 16:0 and cis-9 16:1 fatty acids. FIG. 6C provides the g/100 g fatty acids of 18:0 and cis-9 18:1 fatty acids. FIG. 6D provides the g/100 g fatty acids of trans-11 18:1, and cis-9, trans-11 CLA fatty acids. Diet regimens A and B contained 0.37% VA while regimens C and D contained 1.60% VA. Diet regimens B and D also contained 0.40% sterculic oil, as detailed in Table 7. Data are the means for 9 rats per treatment group. The SEM ranged from 0.01 to 0.06, 0.12 to 0.31, 0.13 to 0.31, and 0.06 to 0.08 for data in FIGS. 6A, B, C, and D, respectively.

### DETAILED DESCRIPTION OF THE INVENTION

According to the invention, vaccenic acid (trans-11 octadecenoic acid) can prevent or reduce the spread and incidence of cancer.

Vaccenic Acid (VA)

Vaccenic acid is trans-11 octadecenoic acid, having the following structure.

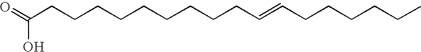

In some embodiments, a triacylglycerol having one or more vaccenic acid moieties is used in the invention. For example, a triacylglycerol with three vaccenic acid moieties is shown below.

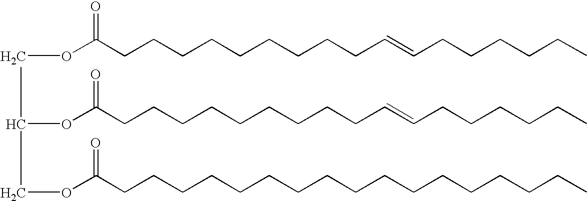

The conversion of vaccenic acid to cis-9, trans-11 conjugated linoleic acid by Δ⁹-desaturase occurs in several species, including rats and humans. According to the invention, increased dietary intake of vaccenic acid provides protection against the development and spread of cancerous tissues. Supplemental vaccenic acid can also improve tissue accumulation of vaccenic acid and CLA. Thus, vaccenic acid has the potential to produce health benefits beyond that associated with traditional fatty acids, particularly trans fatty acids.

Using a biomedical model of chemically-induced mammary carcinogenesis in rats, the inventors have demonstrated that dietary vaccenic acid reduced tumor incidence and tumor number. The prevention of tumor formation was related to dietary supply of vaccenic acid in a dose-dependent manner. Further, vaccenic acid was effective when supplied to the diet as a component of a natural food (e.g. butter) in its naturally occurring chemical form (triglyceride containing fatty acid esters). Dietary vaccenic acid increased the tissue content of both vaccenic acid and cis-9, trans-11 octadecadienoic acid.

The experiments described herein were designed to examine the effects of increasing dietary levels of vaccenic acid and CLA on chemically-induced mammary carcinogenesis in rats. Both fatty acids were provided in the diet as a natural component in butter fat. The overall dietary treatment scheme was aimed at evaluating the modulation of mammary cancer risk by providing (a) small increases of CLA in the presence of a low level of vaccenic acid and (b) more substantial increases of vaccenic acid against a background of low levels of CLA. As expected, small increases in dietary CLA at the low end of the CLA dose-response range did not result in a significant reduction of tumorigenesis. In contrast, there was a distinct and marked inhibitory response when vaccenic acid was added to the diet, and this response was dose-dependent. Fatty acid analysis showed that dietary consumption of vaccenic acid resulted in a dose-dependent increase in the accumulation of vaccenic acid in the mammary fat pad, which was accompanied by a parallel decrease in tumor formation in the mammary gland. The finding confirms that dietary supplementation with vaccenic acid is important for cancer prevention.

Vaccenic acid may inhibit cancer by a variety of mechanisms. One possible mechanism is that vaccenic acid may have a direct effect on tumorigenesis. Another possibility may be that some of the inhibitory effect of vaccenic acid is due to increased tissue content of CLA. Evidence is provided herein that vaccenic acid may increase the tissue content of cis-9, trans-11 linoleic acid.

Treatment with Vaccenic Acid

According to the invention, vaccenic acid can be employed to prevent or reduce the spread and incidence of cancer. Also according to the invention, vaccenic acid (trans-11 octadecenoic acid) can increase the accumulation of vaccenic acid in tissues. The accumulation of CLA is also increased by increased intake of vaccenic acid. Increased amounts of vaccenic acid can be ingested by any suitable means. For example, the compositions and/or foods described herein that contain high or increased levels of vaccenic acid can be ingested.

The terms “cancer” and “cancerous” refer to or describe a physiological condition in mammals that is characterized by unregulated cell growth. As used herein, the term “neoplastic” means characterized by abnormal tissue that shows partial or complete lack of structural organization and functional coordination with normal tissue, and usually forms a distinct mass which may be either benign or malignant.

Examples of cancer include but are not limited to, carcinoma, lymphoma, blastoma, sarcoma, and leukemia. Other examples of such cancers include melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate carcinoma, adenocarcinoma, ovarian adenocarcinoma, renal adenocarcinoma, mammary adenocarcinoma, lung adenocarcinoma, pancreatic adenocarcinoma and non-seminomal testis carcinoma tissues. In some embodiments, the vaccenic compositions are used to prevent and treat breast cancer.

In other embodiments of this invention, the inventive compositions and methods may be used in combination with conventional cancer chemotherapy. Treatment with vaccenic acid may increase the sensitivity of the tumor to conventional cancer chemotherapy and result in greater effectiveness of the conventional cancer chemotherapy drug. For example, the method of this invention can be complemented by a conventional radiation therapy or chemotherapy. Thus, in one embodiment of this invention, the method of this invention comprises administering to a patient a composition containing vaccenic acid and another anticancer agent. Treatment by vaccenic acid can also be conducted along with the treatment with another anticancer agent to increase the effectiveness of the anticancer agent.

Any anticancer agents known in the art can be used in this invention so long as it is pharmaceutically compatible with the vaccenic acid. By “pharmaceutically compatible” it is intended that the other anticancer agent will not interact or react with the vaccenic acid composition, directly or indirectly, in such a way as to adversely affect the effect of the treatment of cancer, or to cause any significant adverse side reaction in the patient.

Exemplary anticancer agents known in the art include busulphan, chlorambucil, hydroxyurea, ifosfamide, mitomycin, mitotane, chlorambucil, mechlorethamine, carmustine, lomustine, cisplatin, carmustine, herceptin, carboplatin, cyclophosphamide, nitrosoureas, fotemustine, vindescine, etoposide, daunorubicin, adriamycin, paclitaxel, docetaxel, streptozocin, dactinomycin, doxorubicin, idarubicin, plicamycin, pentostatin, mitotoxantrone, valrubicin, cytarabine, fludarabine, floxuridine, clardribine, methotrexate, mercaptopurine, thioguanine, capecitabine, irinotecan, dacarbazine, asparaginase, gemcitabine, altretamine, topotecan, procarbazine, vinorelbine, pegaspargase, vincristine, rituxan, vinblastine, tretinoin, teniposide, fluorouracil, melphalan, bleomycin, salicylates, aspirin, piroxicam, ibuprofen, indomethacin, naprosyn, diclofenac, tolmetin, ketoprofen, nambuetone, oxaprozin, doxirubicin, nonselective cycclooxygenase inhibitors such as nonsteroidal anti-inflammatory agents (NSAIDS), and selective cyclooxygenase-2 (COX-2) inhibitors.

The anticancer agent used can be administered simultaneously in the same pharmaceutical preparation with the vaccenic acid composition. The anticancer agent can also be administered at about the same time but by a separate administration. Alternatively, the anticancer agent can be administered at a different time from the administration of the vaccenic acid composition. One of skill in the art can readily determine the best manner of administration, in view of the present disclosure.

The methods of this invention are particularly useful in treating humans. Also, the methods of this invention are suitable for treating cancers in animals, especially mammals such as canine, feline, bovine, porcine, and other animals.

The active compounds of this invention are administered in a pharmaceutically acceptable carrier through any appropriate routes such as parenteral, intravenous, oral, intradermal, subcutaneous, or topical administration. The active compounds of this invention are administered at a therapeutically effective amount to achieve the desired therapeutic effect without causing any serious adverse effects in the patient treated.

Vaccenic acid can be incorporated into triglycerides to facilitate accumulation in fatty tissues such as the mammary gland. Dietary vaccenic acid may have direct effects on mammary epithelial cells, including decreased proliferation and induction of apoptosis. For example, dietary vaccenic acid may prevent the conversion of mammary stromal stem cells to endothelial cells, and inhibit angiogenesis.

Vaccenic Acid Compositions

The present invention contemplates the use of vaccenic acid and derivatives of vaccenic acid. For example, vaccenic acid may be free or bound through ester linkages or provided in the form of an oil containing vaccenic acid triglycerides. In these embodiments, the triglycerides may be partially or wholly comprised of vaccenic acid attached to a glycerol backbone. The vaccenic acid may also preferably be provided as a methylester or ethylester. Furthermore, the vaccenic acid may be in the form of a non-toxic salt, such as a potassium or sodium salt (e.g., a salt formed by reacting chemically equivalent amounts of the free acids with an alkali hydroxide at a pH of about 8 to 9).

Vaccenic acid is produced as an intermediate in rumen biohydrogenation of 18-carbon polyunsaturated fatty acids. Thus, vaccenic acid is a naturally occurring fatty acid present in milk fat and body fat from ruminants. However, the amount of vaccenic acid in dairy products and meat is quite low. Increased amounts of vaccenic acid can be obtained by manipulating the diets of cattle and dairy cows as described in Bauman et al. (2000) J. Dairy Sci. 83: 2422-2425. In particular, when ruminants are fed diets having increased vegetable and fish oils, the ruminants produce milk and meat that is enriched in vaccenic acid. For example, ruminants fed a diet of 2% sunflower oil and 1% fish oil produced a milk fat that was enriched with vaccenic acid. In some embodiments, the ruminants are fed diets containing about 0.2% to about 5% vegetable oil and/or about 0.1% to about 5% fish oil. The vegetable oil can be safflower oil, corn oil, sunflower oil, soybean oil, flax oil, sesame oil or another vegetable oil.

Milk containing increased amounts of vaccenic acid can be collected and processed to manufacture butter or other dairy products as described in Bauman et al. (2000) J. Dairy Sci. 83: 2422-2425.

In some embodiments, a vaccenic acid triacylglycerol is synthesized that has at least one vaccenic acid moiety. The presence of vaccenic acid in triacylglycerols may be confirmed by H¹ NMR. In some embodiments, the triacylglycerols contain at least about 33% or 66% or at least about 50% or 95% or more of trans-11 octadecenoic acid. In some embodiments, the resultant triacylglycerol is not purified further to remove all levels of phosphatidyl and sterol residues. But those levels remaining from isomerization of sunflower and safflower oils will be adequate for commercial applications involving safe, edible products in feed and food. In other embodiments, the triacylglycerol is further purified, for example, by molecular distillation.

An immobilized Candida antarctica lipase can be employed for esterification in a manner similar to that described in U.S. Pat. No. 6,524,527. The esterification reaction is conducted at about 50° to about 75° C., or at about 65° C., in the absence of any solvent. A vacuum can be employed in order to remove the co-produced water or alcohols (from esters) upon formation. This shifts the triacylglycerol production to completion and ensures a highly pure product virtually free of any mono- and diacylglycerols in essentially quantitative yields. Stoichiometric amounts of free fatty acids may be used, i.e. 3 molar equivalents as based on glycerol or 1 molar equivalent as based on number of mol equivalents of hydroxyl groups present in the glycerol moiety. Only 10% dosage of lipase as based on total weight of substrates is needed, which can be used a number of times. Also, a slight excess (<5/5) of free fatty acids may be used in order to speed the reaction and ensure completion of the reaction.

At the initiation of the reaction, the 1- or 3-mono-acyglyeride is formed first, followed by the 1,3 diacylglyeride, and finally the triglyceride at the more extended reaction times. The mono- and diacylglyerides are useful intermediates in that they manifest biological activity, but have greater solubility in aqueous cellular environments and can participate in alternative molecular synthetic pathways such as synthesis of phospholipids or other functional lipids. In contrast, triglycerides are frequently deposited intact in cell membranes or storage vesicles. Thus, the administration of vaccenic acid in mono-, di- or triglycerol form rather than free fatty acid or ester, may influence the mode and distribution of uptake, metabolic rate and structural or physiological role of the vaccenic acid component.

The vaccenic acid compositions of the invention may be provided in a variety of forms. In some embodiments, administration is oral. The production of pure vaccenic acid pills for the marketplace is contemplated by the invention. On the other hand, vaccenic acid-enriched food also is contemplated for delivering these anticancer agents to the general public. The vaccenic acid compositions may therefore be formulated with suitable carriers such as starch, sucrose or lactose in tablets, pills, dragees, capsules, solutions, liquids, slurries, suspensions and emulsions. The vaccenic acid compositions can also contain antioxidants, including, but not limited to Controx, Covi-OX, lecithin, vitamin E and oil soluble forms of vitamin C (ascorbyl palmitate). The vaccenic acid compositions may be provided in aqueous solutions, oily solutions, oil-in-water suspensions, water-in-oil suspensions or in any of the other forms generated by one of skill in the art. The tablet or capsule of the present invention may be coated with an enteric coating which dissolves at a pH of about 6.0 to 7.0. A suitable enteric coating that dissolves in the small intestine but not in the stomach is cellulose acetate phthalate. In some embodiments, the vaccenic acid composition is provided as soft gelatin capsules containing 750 mg 80% vaccenic acid.

The vaccenic acid compositions may also be administered by any of a number of other routes, including, but not limited to, intravenous, intramuscular, intra-arterial, intramedullary, intrathecal, intraventricular, transdermal, subcutaneous, intraperitoneal, intranasal, enteral, topical, sublingual or rectal means. Further details on techniques for formulation and administration may be found in the latest edition of Remington's Pharmaceutical Sciences (Maack Publishing Co., Easton, Pa.).

An effective amount of vaccenic acid may also be provided as a supplement in various food and drink products for human consumption. The vaccenic acid compositions can also be incorporated into animal feeds. For the purposes of this application, food products containing vaccenic acid means any natural, processed, diet or non-diet food product to which exogenous vaccenic acid has been added. The vaccenic acid may be added in the form of free fatty acids, esters of vaccenic acid, or oils containing partial or whole triglycerides of vaccenic acid.

Therefore, vaccenic acid may be directly incorporated into various prepared food products, including, but not limited to diet drinks, diet bars, cookies, health bars, salad dressings, sauces, creams, whipped cream, ice cream, butter, milk shakes, health drinks, powdered drinks, powdered cake mixes, powdered snack bar mixes (e.g. brownie mixes), margarine, cooking oil or lard, health supplements, prepared frozen meals, candy, snack such as crackers and chips, prepared meat products, milk, cheese, yogurt and any other fat or oil containing foods.

Furthermore, vaccenic acid compositions can contain some volatile organic compounds, but not so much as to cause the taste and smell of food products containing the vaccenic acid to be adversely effected. It is contemplated that the food products of the present invention that contain vaccenic acid compositions having less than 100 ppm volatile organic compounds, and preferably less than 5 ppm volatile organic compounds, are superior in taste and smell to food products containing higher levels of volatile organic compounds and will be preferred in blind taste and smell tests. Accordingly, some embodiments of the present invention provide a food product containing a vaccenic acid, wherein the vaccenic acid has a sufficiently low volatile organic acid compound concentration so that taste and smell of the food product is not affected.

The labeling of the trans fatty acid (TFA) content of foods has received renewed attention because of the relationship of dietary intake with increases in plasma LDL cholesterol and the risk of coronary heart disease. Institute of Medicine (2002) Letter report on dietary reference intakes for trans fatty acids. Other workers have challenged the appropriateness of grouping all TFA into a single entity for food labeling and used cis-9, trans-11 conjugated linoleic acid isomers as examples of TFA for which beneficial effects have been identified. Belury, M. (2002) Not all trans-fatty acids are alike: What consumers may lose when we oversimplify nutrition facts. J. Am. Diet. Assoc. 102: 1606.

The present invention demonstrates the beneficial effects of trans-11 octadecenoic acid on tumorigenesis in a rodent model, and further indicates that certain trans octadecenoic acid isomers can also have beneficial effects. Food products containing partially hydrogenated vegetable oils (PHVO) are the major dietary source of TFA in the U.S. (about 90% of total). These are predominantly trans-18:1 acids and the partial hydrogenation of vegetable oil produces a Gaussian distribution of trans 18:1 isomers that centers on trans-9, trans-0, and trans-11 isomers. Emken, E. A. (1995) Am. J. Clin. Nutr. 62: 659S-669S; Craig-Schmidt, M. C. (1992) Fatty acid isomers in foods. In: Fatty Acids in Foods and their Health Implications (Chow, C. K., ed.), pp.365-398. Marcel Dekker Inc., New York, N.Y. The remainder of dietary TFA comes from food products derived from ruminants, and in this case the major isomer is vaccenic acid. Emken (1995). The present study demonstrates that when vaccenic acid was supplied in the diet as a natural component, clear benefits may result, such as a reduction in the incidence of mammary tumors. Interestingly, epidemiological studies have observed a relationship between coronary heart disease risk and dietary intake of TFA from vegetable sources, but no such relationship exists for TFA intake from animal derived foods. Willet et al. (1993) Lancet 341: 581-585; Pietinen et al. (1997) Am. J. Epidem. 145: 876-887; Gillman et al. (1997) Epidem. 8: 144-149.

The Examples further illustrate the invention but are not intended to limit it in any way.

### EXAMPLE 1

### Supplemental Vaccenic Acid Prevents Tumor Formation

This Example illustrates that the incidence of tumors in rats is significantly decreased, in a dose-dependent manner, when increasing amounts of vaccenic acid are provided in the diet.

Materials and Methods

Production of experimental butterfats. The dietary treatments employed in the rodent carcinogenesis experiment were designed to differ in the content of vaccenic acid and cis-9, trans-11 CLA provided as a natural food. This was achieved by preparing diet formulations using combinations of butter from two sources. The butter sources were produced by manipulating the diets of dairy cows with natural feed ingredients as described in Bauman et al. (2000) J. Dairy Sci. 83: 2422-2425. One group of cows was fed a corn-based total mixed ration to produce a control milk fat and a second group of cows was fed the same total mixed ration supplemented with 2% sunflower oil and 1% fish oil to produce a milk fat that was enriched with vaccenic acid and cis-9, trans-11 CLA. Milk was collected and processed from these two groups of cows to manufacture butter as described in Bauman et al. (2000) J. Dairy Sci. 83: 2422-2425. The fatty acid composition of the two sources of butter is presented in Table 1.

TABLE 1
Fatty acid composition of control butter fat and
high vaccenic acid-enriched (VA) butter fat
in g/100 g fatty acids

| | Control | VA-enriched |
|---|---|---|
| Fatty Acid | Butter | Butter |
| 4:0 | 4.12 | 3.69 |
| 6:0 | 2.04 | 1.81 |
| 8:0 | 1.10 | 0.99 |
| 10:0 | 2.27 | 2.10 |
| 12:0 | 2.50 | 2.31 |
| 14:0 | 8.87 | 8.67 |
| 14:1, cis-9 | 0.74 | 0.63 |
| 15:0 | 0.75 | 0.82 |
| 16:0 | 27.62 | 23.01 |
| 16:1, cis-9 | 1.52 | 1.33 |
| 17:0 | 0.47 | 0.45 |
| 18:0 | 11.85 | 5.09 |
| 18:1, trans-6 to 8 | 0.46 | 1.21 |
| 18:1, trans-9 | 0.36 | 0.84 |
| 18:1, trans-10 | 0.67 | 2.68 |
| 18:1, trans-11 | 1.30 | 16.28 |
| 18:1, trans-12 | 0.77 | 2.48 |
| 18:1, cis-9 | 25.35 | 14.23 |
| 18:2, cis-9, cis-12 | 3.33 | 2.93 |
| CLA, cis-9, trans-11 | 0.51 | 3.76 |
| 18:3, cis-9, cis-12, cis-15 | 0.41 | 0.46 |
| 20:0 | 0.14 | 0.13 |
| Others | 2.85 | 4.10 |

There were minor differences in concentrations of several fatty acids between the two butter sources, but there were major differences in the content of vaccenic acid and CLA.

Protocol of animal treatment. Female Sprague-Dawley rats were purchased from Charles River Breeding Laboratories (Raleigh, N.C.) at 45 days of age and all subsequent procedures were approved by Roswell Park Cancer Institute Animal Care and Use Committee. Rats were fed the AIN-76 basal diet for 1 week to acclimatize them to the powdered diet. All animals were injected with a single dose of methylnitrosourea (MNU: 50 mg/kg body weight) intraperitoneally at 52 days of age for the induction of mammary tumors. Immediately after MNU administration, a total of 210 rats were divided into 7 groups of 30 each and maintained on different dietary treatments for the following 24 weeks. Throughout this period, rats were palpated weekly to determine the appearance, size, and location of mammary tumors. The experiment was terminated at 24 weeks after MNU administration. All tumors were excised and fixed for histological examination and only confirmed carcinomas are reported in data summaries. Randomly-selected subsets of nine rats from each treatment were necropsied. The tumor-free inguinal mammary fat pad, a portion of the liver, and plasma were retrieved. Tissue and plasma samples were immediately frozen in liquid nitrogen and then stored at −80° C. until fatty acid analysis.

Diet formulation and feeding. All seven diets contained a total of 10% butter fat by weight and this was derived from the two butter sources either alone or in combination as shown in Table 2.

TABLE 2
Design of diets containing control butter fat
and/or vaccenic acid-enriched butter fat

| | Diet Proportion | | | | |
|---|---|---|---|---|---|
| | | VA- | | Fatty Acid Content | |
| | Control | enriched | Synthetic CLA | 18:1 | CLA |
| | Butter | Butter | cis-9, trans-11 | trans-11 | cis-9, trans-11 |
| Diet | Diet % | | | Diet % | |
| A | 10 | | | 0.13 | 0.05 |
| B | 10 | | 0.13 | 0.13 | 0.18 |
| C | 10 | | 0.19 | 0.13 | 0.24 |
| D | 10 | | 0.32 | 0.13 | 0.37 |
| E | 6 | 4 | | 0.73 | 0.18 |
| F | 4 | 6 | | 1.00 | 0.24 |
| G | | 10 | | 1.60 | 0.37 |

Diet A represented the control group that contained a low level of both CLA and vaccenic acid. By using different proportions of the control butter and the vaccenic acid-enriched butter, diets E, F and G were formulated with increasing concentrations of vaccenic acid at 0.73%, 1% or 1.6%. Because the two sources of butter contained different levels of CLA, diets B, C and D were supplemented with synthetic cis-9, trans-11 conjugated linoleic acid (Natural, Hovbebygda, Norway; 90% purity) so that the total CLA content in diet B matched CLA content in diet E, diet C matched diet F, and diet D matched diet G. All diets contained the same amounts of casein, dextrose, mineral mix, vitamin mix, alphacel, DL-methionine and choline bitartrate as described in Ip et al. (1999) J. Nutr. 129: 2135-2142. All animals were fed ad libitum with food and water available at all times. Fresh food was given every two days.

Fatty acid analysis. Samples of liver and mammary fat pad maintained at −80° C. were pulverized at liquid nitrogen temperature. Total lipids were then extracted from pulverized tissues and plasma using a mixture of hexane and isopropanol and following the procedure of Hara and Radin (1978) Anal. Bioch. 90: 420-426. Fatty acids were methylated according to a modified method related to that described in Christie (1982) Lipid Res. 23: 1072-1075. For fat pad lipids, 40 mg were dissolved in 2.0 mL hexane and 40 μL methyl acetate. Forty μL of methylation reagent (1.0 mol/L sodium methoxide in methanol) was added, the solution thoroughly mixed, and allowed to react at room temperature for 10 min. The reaction was then terminated by the addition of 60 μL of 0.26 mol/L oxalic acid in diethyl ether. Several grains of anhydrous calcium chloride were added and the mixture was incubated at room temperature for 1 hour. An aliquot of the clear hexane supernatant was removed for analysis by gas chromatography following centrifugation at 2,400×g, 4° C. for 5 min. Plasma and liver samples were processed in a similar manner to mammary fat pad. However, because these typically yielded far less lipid, quantities of all reagents for methylation were adjusted to compensate for the reduced yield.

Fatty acid methyl esters were analyzed by gas chromatography (Hewlett Packard GC system 6890+with flame ionization detector) using a CP-Sil 88 capillary column (100 m×0.25 mm i.d. with 0.2 μm film thickness; Varian Inc.). A programmed temperature run was used to separate fatty acid methyl esters. Inlet and detector temperatures were 250° C. The oven temperature was initially 80° C., then ramped 2° C./min to 190° C. and held 20 min until ramped 5° C./min to 215° C. and held for 15 min. The split ratio was 100:1 and hydrogen was used as the carrier gas at 1.0 mL/min. Fatty acid methyl ester standards were used to identify sample fatty acid methyl esters (Nu-Chek Prep; Elysian, Minn.).

Statistical analyses. Fatty acid composition data were analyzed statistically by the General Linear Model procedure of SAS (SAS Inc., Cary, N.C.). For treatments A, B, C, and D, a single factor ANOVA was used to identify the effect of treatment. Differences between treatment means were identified using the PDIFF option of the LSMeans command. Linear contrasts were used to determine differences between treatments B and E, C and F, and D and G. Treatment effects and differences between means were considered significant when P<0.05. Tumor incidence was compared by χ-square analysis using the Frequency procedure of SAS. Total tumor numbers were compared by frequency distribution analysis. See Horvath (1983) Cancer Res. 43: 5335-5341.

Results

The overall dietary treatment scheme was aimed at evaluating the modulation of mammary cancer risk by (a) small increases of CLA in the presence of a low level of vaccenic acid and (b) more substantial increases of vaccenic acid against a background of low levels of CLA. Therefore, comparisons between treatments A, B, C, and D examine the effect of increasing dietary cis-9, trans-11 CLA concentration while maintaining a constant vaccenic acid concentration. Comparisons between treatments with identical cis-9, trans-11 CLA concentrations, but differing vaccenic acid concentrations involve treatments B vs. E, C vs. F, and D vs. G (Table 2).

Modulation of mammary cancer risk The mammary carcinogenesis data are summarized in Table 3.

TABLE 3
Bioassay of mammary cancer prevention in rats
fed increasing levels of CLA and/or vaccenic acid

| Treatment¹ | Tumor Incidence (%) | Total Tumors |
|---|---|---|
| A (0.13% VA, 0.05% CLA) | 93 | 91 |
| B (0.13% VA, 0.18% CLA) | 93 | 85 |
| C (0.13% VA, 0.24% CLA | 83 | 74 |
| D (0.13% VA, 0.37% CLA) | 77 (P = 0.15)² | 65 (P < 0.05) |
| E (0.73% VA, 0.18% CLA) | 70 (P < 0.05)³ | 63 (P < 0.05) |
| F (1.0% VA, 0.24% CLA) | 47 (P < 0.01)⁴ | 49 (P < 0.05) |
| G (1.6% VA, 0.37% CLA) | 40 (P < 0.01)⁵ | 36 (P < 0.05) |

¹Diets differed in content of CLA (Treatments A, B, C, and D) and vaccenic acid (B vs. E, C vs. F, and D vs. G) as detailed in Table 2.
²P-value for Single Factor ANOVA comparing treatments A, B, C, and D.
³P-value for linear contrast comparing treatments B and E.
⁴P-value for linear contrast comparing treatments C and F.
⁵P-value for linear contrast comparing treatments D and G.

Although there was a progressive numerical decline in tumor incidence with increasing dietary intake of cis-9, trans-11 CLA (treatments A, B, C and D), the decrease was not statistically significant. It should be noted that in this design, the dietary CLA level ranged from 0.05% in treatment A to 0.37% in treatment D. This range is at the low end of the CLA dose response curve based on the available historical data.

In contrast, the reduction in tumor incidence was more pronounced with increasing dietary vaccenic acid (treatments E, F and G). Against this backdrop of the small increases of CLA, there was a distinct inhibitory response to vaccenic acid which was dose-dependent. The effect of vaccenic acid was magnified in this experiment because the dose range was broadened when compared to that of CLA; additionally, the entire dose range of vaccenic acid was shifted a bit more to the right. The last column in Table 3 shows the total tumor data in each group. The tumor yield data closely paralleled the tumor incidence data, thus further confirming the consistency of the results.

Fatty acid analysis of tissue and plasma lipids. The fatty acid composition data for liver, plasma, and mammary fat pad are presented in Tables 4, 5, and 6, respectively.

TABLE 4
Composition of fatty acids from liver lipids of rats fed increasing amounts
of vaccenic Acid (trans-11 18:1) and conjugated linoleic acid (CLA)

| | Treatment¹ | | | | | Treatment¹ | | | | | |
|---|---|---|---|---|---|---|---|---|---|---|---|
| | A | B | C | D | | E | F | G | | | |
| Fatty acid | g/100 g fatty acids | | | | P² | g/100 g fatty acids | | | P³ | P⁴ | P⁵ |
| 14:0 | 0.56 | 0.54 | 0.58 | 0.67 | | 0.52 | 0.48 | 0.42 | | | * |
| 14:1, cis-9 | 0.03 | 0.05 | 0.05 | 0.06 | | 0.03 | 0.02 | 0.01 | | | ** |
| 15:0 | 0.14 | 0.14 | 0.13 | 0.15 | | 0.16 | 0.15 | 0.14 | | | |
| 16:0 | 15.92 | 15.34 | 15.64 | 16.64 | | 16.13 | 14.88 | 13.91 | | | * |
| 16:1, cis-9 | 2.10 | 2.08 | 2.16 | 2.42 | | 1.88 | 1.78 | 1.55 | | | * |
| 17:0 | 0.30 | 0.31 | 0.29 | 0.31 | | 0.33 | 0.36 | 0.36 | | ** | ** |
| 18:0 | 24.19 | 23.88 | 23.00 | 21.82 | | 22.61 | 23.42 | 23.98 | | | |
| 18:1, trans-6 | 0.09 | 0.08 | 0.08 | 0.08 | | 0.13 | 0.15 | 0.19 | ** | ** | ** |
| to 8 | | | | | | | | | | | |
| 18:1, trans-9 | 0.11 | 0.12 | 0.12 | 0.13 | | 0.17 | 0.20 | 0.25 | ** | ** | ** |
| 18:1, trans- | 0.05 | 0.05 | 0.06 | 0.06 | | 0.13 | 0.17 | 0.25 | ** | ** | ** |
| 10 | | | | | | | | | | | |
| 18:1, trans- | 0.19 | 0.22 | 0.23 | 0.24 | | 1.14 | 1.66 | 2.63 | ** | ** | ** |
| 11 | | | | | | | | | | | |
| 18:1, trans- | 0.23 | 0.28 | 0.30 | 0.28 | | 0.35 | 0.40 | 0.41 | ** | ** | ** |
| 12 | | | | | | | | | | | |
| 18:1, cis-9 | 14.46 | 14.47 | 14.97 | 17.20 | | 14.39 | 12.73 | 10.73 | | | ** |
| 18:2, cis-9, | 3.42 | 3.84 | 3.69 | 3.76 | | 4.51 | 4.49 | 4.97 | ** | ** | ** |
| cis-12 | | | | | | | | | | | |
| CLA, cis-9, | 0.09^{b} | 0.12^{b} | 0.13^{b} | 0.19^{a} | ** | 0.61 | 0.76 | 1.10 | ** | ** | ** |
| cis-11 | | | | | | | | | | | |
| 18:3, all cis | 0.07 | 0.09 | 0.07 | 0.08 | | 0.06 | 0.09 | 0.05 | | | |
| 6, 9, 12 | | | | | | | | | | | |
| 18:3, all cis | 0.05 | 0.18 | 0.17 | 0.11 | | 0.19 | 0.09 | 0.11 | | | |
| 9, 12, 15 | | | | | | | | | | | |
| 20:0 | 0.06 | 0.04 | 0.03 | 0.02 | | 0.01 | 0.02 | 0.06 | | | * |
| 20:1, cis-11 | 0.09 | 0.06 | 0.08 | 0.08 | | 0.07 | 0.06 | 0.08 | | | |
| 20:3, all cis | 1.14 | 1.16 | 1.03 | 1.03 | | 1.21 | 1.20 | 1.47 | | | ** |
| 8, 11, 14 | | | | | | | | | | | |
| 20:4, all cis | 19.15 | 19.45 | 19.58 | 17.58 | | 16.66 | 17.12 | 16.39 | * | * | |
| 5, 8, 11, 14 | | | | | | | | | | | |
| 20:5, all cis | 0.48 | 0.52 | 0.43 | 0.50 | | 0.71 | 0.74 | 1.06 | * | ** | ** |
| 5, 8, 11, 14, | | | | | | | | | | | |
| 17 | | | | | | | | | | | |
| 22:4, all cis | 0.21 | 0.23 | 0.22 | 0.23 | | 0.18 | 0.27 | 0.17 | | | |
| 7, 10, 13, 16 | | | | | | | | | | | |
| 22:5, all cis | 0.25 | 0.29 | 0.27 | 0.24 | | 0.30 | 0.36 | 0.34 | | * | ** |
| 7, 10, 13, 16, | | | | | | | | | | | |
| 19 | | | | | | | | | | | |
| 22:6, all cis | 8.12 | 7.92 | 7.97 | 7.16 | | 8.52 | 9.62 | 10.83 | | ** | ** |
| 4, 7, 10, 13, | | | | | | | | | | | |
| 16, 19 | | | | | | | | | | | |
| Other | 8.50 | 8.53 | 8.71 | 8.95 | | 9.00 | 8.75 | 8.53 | | | |

* P < 0.05.
** P < 0.01.
¹Diets differed in content of cis-9, trans-11 CLA (Treatments A, B, C, and D) and vaccenic acid (trans-11 18:1); B vs. E, C vs. F, and D vs. G) as detailed in Table 2.
²P-value for Single Factor ANOVA comparing treatments A, B, C, and D.
³P-value for linear contrast comparing treatments B and E.
⁴P-value for linear contrast comparing treatments C and F.
⁵P-value for linear contrast comparing treatments D and G.

TABLE 5
Composition of fatty acids from plasma lipids of rats fed
increasing amounts of vaccenic acid (trans-11 18:1) and
cis-9, trans-11 conjugated linoleic acid (CLA)

| | Treatment¹ | | | | | Treatment¹ | | | | | |
|---|---|---|---|---|---|---|---|---|---|---|---|
| | A | B | C | D | | E | F | G | | | |
| Fatty acid | g/100 g fatty acids | | | | P² | g/100 g fatty acids | | | P³ | P⁴ | P⁵ |
| 12:0 | 0.19^{a} | 0.13^{ab} | 0.12^{b} | 0.09^{b} | * | 0.18 | 0.14 | 0.13 | | | |
| 14:0 | 1.59 | 1.35 | 1.23 | 1.11 | | 1.50 | 1.37 | 1.36 | | | |
| 14:1, cis-9 | 0.17 | 0.12 | 0.12 | 0.11 | | 0.12 | 0.11 | 0.09 | | | |
| 15:0 | 0.29 | 0.28 | 0.26 | 0.25 | | 0.32 | 0.30 | 0.31 | | * | ** |
| 16:0 | 21.75 | 21.37 | 21.49 | 20.94 | | 22.41 | 21.06 | 21.86 | | | |
| 16:1, cis-9 | 3.51 | 3.66 | 3.67 | 3.38 | | 2.90 | 3.02 | 3.26 | | | |
| 17:0 | 0.30 | 0.29 | 0.28 | 0.28 | | 0.35 | 0.36 | 0.34 | ** | ** | ** |
| 18:0 | 14.77 | 14.48 | 14.18 | 14.52 | | 14.53 | 14.67 | 12.00 | | | * |
| 18:1, trans-6 | 0.13 | 0.12 | 0.12 | 0.12 | | 0.21 | 0.23 | 0.29 | ** | ** | ** |
| to 8 | | | | | | | | | | | |
| 18:1, trans-9 | 0.14 | 0.15 | 0.15 | 0.14 | | 0.24 | 0.27 | 0.36 | ** | ** | ** |
| 18:1, trans- | 0.15 | 0.12 | 0.13 | 0.12 | | 0.33 | 0.38 | 0.56 | ** | ** | ** |
| 10 | | | | | | | | | | | |
| 18:1, trans- | 0.31 | 0.28 | 0.27 | 0.26 | | 1.82 | 2.42 | 3.77 | ** | ** | ** |
| 11 | | | | | | | | | | | |
| 18:1, trans- | 0.32 | 0.32 | 0.33 | 0.31 | | 0.46 | 0.57 | 0.79 | | * | ** |
| 12 | | | | | | | | | | | |
| 18:1, cis-9 | 29.59 | 30.55 | 30.97 | 31.76 | | 27.22 | 25.85 | 26.31 | * | ** | ** |
| 18:2, cis-9, | 4.36 | 4.53 | 4.32 | 4.36 | | 5.19 | 4.92 | 4.82 | | | |
| cis-12 | | | | | | | | | | | |
| CLA, , cis-9, | 0.24^{b} | 0.29^{a} | 0.31^{a} | 0.33^{a} | ** | 1.31 | 1.76 | 3.39 | ** | ** | ** |
| cis-11 | | | | | | | | | | | |
| 18:3, all cis | 0.06 | 0.08 | 0.07 | 0.08 | | 0.05 | 0.05 | 0.04 | ** | | ** |
| 6, 9, 12 | | | | | | | | | | | |
| 18:3, all cis | 0.11 | 0.10 | 0.07 | 0.08 | | 0.12 | 0.09 | 0.11 | | | |
| 9, 12, 15 | | | | | | | | | | | |
| 20:0 | 0.03 | 0.03 | 0.03 | 0.02 | | 0.02 | 0.04 | 0.04 | | | |
| 20:1, cis-11 | 0.13 | 0.14 | 0.16 | 0.17 | | 0.15 | 0.13 | 0.16 | | | |
| 20:3, all cis | 0.62 | 0.67 | 0.57 | 0.62 | | 0.73 | 0.69 | 0.71 | | | |
| 8, 11, 14 | | | | | | | | | | | |
| 20:4, all cis | 9.40 | 9.54 | 9.88 | 9.45 | | 8.36 | 8.70 | 6.07 | | | ** |
| 5, 8, 11, 14 | | | | | | | | | | | |
| 20:5, all cis | 0.27 | 0.29 | 0.26 | 0.29 | | 0.37 | 0.33 | 0.40 | | | |
| 5, 8, 11, 14, | | | | | | | | | | | |
| 17 | | | | | | | | | | | |
| 22:4, all cis | 0.09 | 0.09 | 0.07 | 0.04 | | 0.01 | 0.02 | 0.01 | ** | | |
| 7, 10, 13, 16 | | | | | | | | | | | |
| 22:5, all cis | 0.04 | 0.08 | 0.07 | 0.04 | | 0.10 | 0.02 | 0.11 | | | |
| 7, 10, 13, 16, | | | | | | | | | | | |
| 19 | | | | | | | | | | | |
| 22:6, all cis | 2.44 | 2.46 | 2.64 | 2.58 | | 2.92 | 3.52 | 3.14 | | ** | |
| 4, 7, 10, 13, | | | | | | | | | | | |
| 16, 19 | | | | | | | | | | | |
| Other | 9.01 | 8.45 | 8.25 | 8.57 | | 8.08 | 8.99 | 9.56 | | | |

* P < 0.05.
** P < 0.01.
¹Diets differed in content of cis-9, trans-11 CLA (Treatments A, B, C, and D) and vaccenic acid (trans-11 18:1; B vs. E, C vs. F, and D vs. G) as detailed in Table 2.
²P-value for Single Factor ANOVA comparing treatments A, B, C, and D.
³P-value for linear contrast comparing treatments B and E.
⁴P-value for linear contrast comparing treatments C and F.
⁵P-value for linear contrast comparing treatments D and G.

TABLE 6
Composition of fatty acids from mammary fat pad lipids of rats
fed increasing amounts of vaccenic acid (trans-11 18:1) and cis-9,
trans-11 conjugated linoleic acid (CLA)

| | Treatment¹ | | | | | Treatment¹ | | | | | |
|---|---|---|---|---|---|---|---|---|---|---|---|
| | A | B | C | D | | E | F | G | | | |
| Fatty acid | g/100 g fatty acids | | | | P² | g/100 g fatty acids | | | P³ | P⁴ | P⁵ |
| 12:0 | 0.75 | 0.74 | 0.72 | 0.77 | | 0.76 | 0.75 | 0.69 | | | ** |
| 14:0 | 3.99 | 4.01 | 3.90 | 4.07 | | 4.13 | 3.97 | 3.86 | | | * |
| 14:1, cis-9 | 0.48 | 0.47 | 0.46 | 0.46 | | 0.46 | 0.43 | 0.41 | | | ** |
| 15:0 | 0.41 | 0.42 | 0.42 | 0.42 | | 0.45 | 0.44 | 0.46 | | | ** |
| 16:0 | 27.05 | 26.68 | 26.45 | 26.56 | | 25.53 | 25.12 | 24.66 | ** | ** | ** |
| 16:1, cis-9 | 7.12 | 6.91 | 7.16 | 6.68 | | 6.45 | 6.38 | 6.36 | | | |
| 17:0 | 0.28 | 0.29 | 0.29 | 0.29 | | 0.30 | 0.30 | 0.31 | | | |
| 18:0 | 4.16 | 4.19 | 4.06 | 4.31 | | 3.94 | 3.65 | 3.21 | | * | ** |
| 18:1, trans-6 | 0.18 | 0.18 | 0.17 | 0.18 | | 0.28 | 0.31 | 0.39 | ** | ** | ** |
| to 8 | | | | | | | | | | | |
| 18:1, trans-9 | 0.22 | 0.22 | 0.22 | 0.23 | | 0.34 | 0.37 | 0.44 | ** | ** | ** |
| 18:1, trans- | 0.25 | 0.25 | 0.26 | 0.25 | | 0.51 | 0.62 | 0.89 | ** | ** | ** |
| 10 | | | | | | | | | | | |
| 18:1, trans- | 0.36 | 0.37 | 0.37 | 0.38 | | 2.15 | 2.75 | 4.17 | ** | ** | ** |
| 11 | | | | | | | | | | | |
| 18:1, trans- | 0.40 | 0.39 | 0.39 | 0.38 | | 0.74 | 0.88 | 1.10 | ** | ** | ** |
| 12 | | | | | | | | | | | |
| 18:1, cis-9 | 39.27 | 39.45 | 39.64 | 39.74 | | 35.84 | 33.59 | 31.22 | ** | ** | ** |
| 18:2, cis-9, | 6.22 | 5.94 | 6.22 | 5.71 | | 5.50 | 6.29 | 6.43 | | | |
| cis-12 | | | | | | | | | | | |
| CLA | 0.48^{c} | 0.57^{b} | 0.59^{b} | 0.66^{a} | ** | 2.21 | 2.85 | 4.14 | ** | ** | ** |
| 18:3, all cis | 0.04 | 0.04 | 0.04 | 0.04 | | 0.03 | 0.03 | 0.04 | * | | |
| 6, 9, 12 | | | | | | | | | | | |
| 18:3, all cis | 0.29 | 0.27 | 0.29 | 0.26 | | 0.24 | 0.27 | 0.29 | | | |
| 9, 12, 15 | | | | | | | | | | | |
| 20:0 | 0.06 | 0.06 | 0.06 | 0.07 | | 0.07 | 0.06 | 0.06 | | | |
| 20:1, cis-11 | 0.14 | 0.13 | 0.14 | 0.14 | | 0.14 | 0.15 | 0.14 | | | |
| 20:3, all cis | 0.03 | 0.03 | 0.03 | 0.03 | | 0.03 | 0.03 | 0.04 | | | |
| 8, 11, 14 | | | | | | | | | | | |
| 20:4, all cis | 0.26 | 0.25 | 0.27 | 0.25 | | 0.27 | 0.27 | 0.25 | | | |
| 5, 8, 11, 14 | | | | | | | | | | | |
| 20:5, all cis | n.d. | n.d. | n.d. | n.d. | | n.d. | n.d. | n.d. | | | |
| 5, 8, 11, 14, | | | | | | | | | | | |
| 17 | | | | | | | | | | | |
| 22:4, all cis | 0.03 | 0.03 | 0.04 | 0.04 | | 0.03 | 0.02 | 0.03 | | ** | |
| 7, 10, 13, 16 | | | | | | | | | | | |
| 22:5, all cis | 0.01^{b} | 0.01^{b} | 0.03^{a} | 0.02^{b} | ** | 0.02 | 0.01 | 0.03 | | * | * |
| 7, 10, 13, 16, | | | | | | | | | | | |
| 19 | | | | | | | | | | | |
| 22:6, all cis | 0.05 | 0.04 | 0.04 | 0.04 | | 0.06 | 0.07 | 0.07 | ** | ** | ** |
| 4, 7, 10, 13, | | | | | | | | | | | |
| 16, 19 | | | | | | | | | | | |
| Other | 7.47 | 8.04 | 7.75 | 8.03 | | 9.51 | 10.37 | 10.30 | ** | ** | ** |

* P < 0.05.
** P < 0.01.
n.d. not detected.
¹Diets differed in content of cis-9, trans-11 CLA (Treatments A, B, C, and D) and vaccenic acid (trans-11 18:1; B vs. E, C vs. F, and D vs. G) as detailed in Table 2.
²P-value for Single Factor ANOVA comparing treatments A, B, C, and D.
³P-value for linear contrast comparing treatments B and E.
⁴P-value for linear contrast comparing treatments C and F.
⁵P-value for linear contrast comparing treatments D and G.

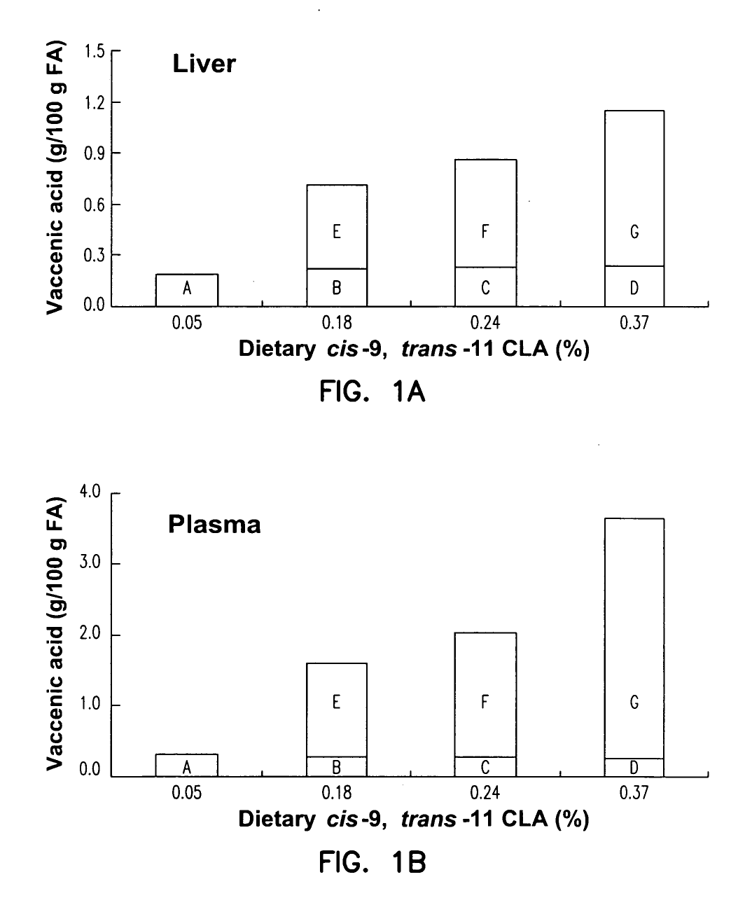

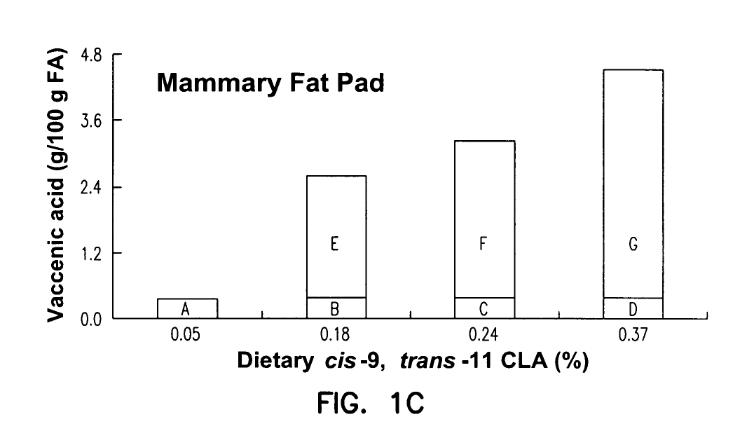

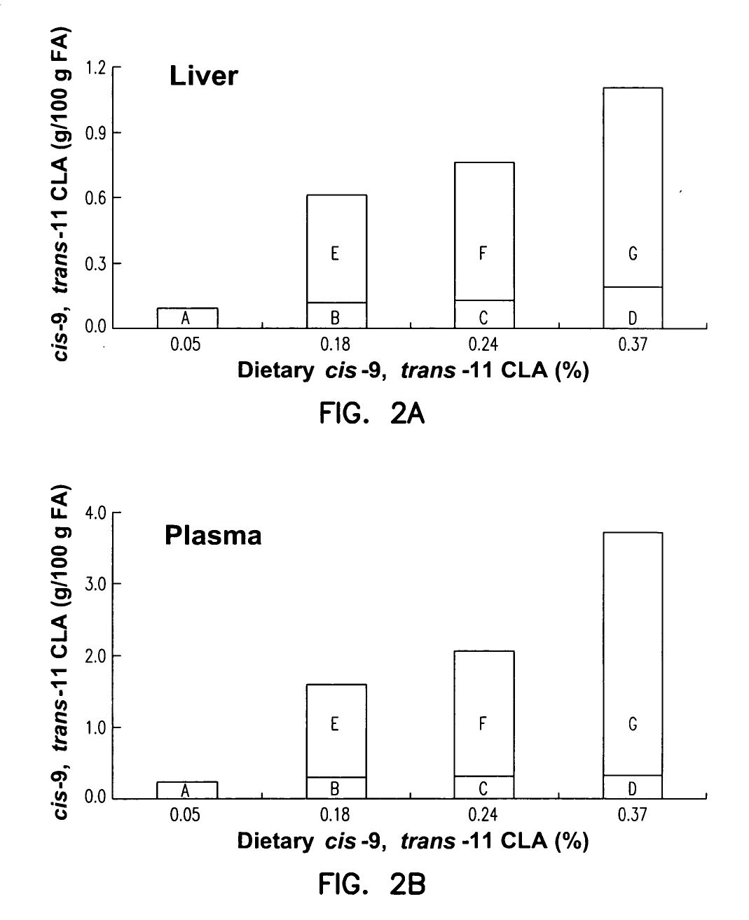

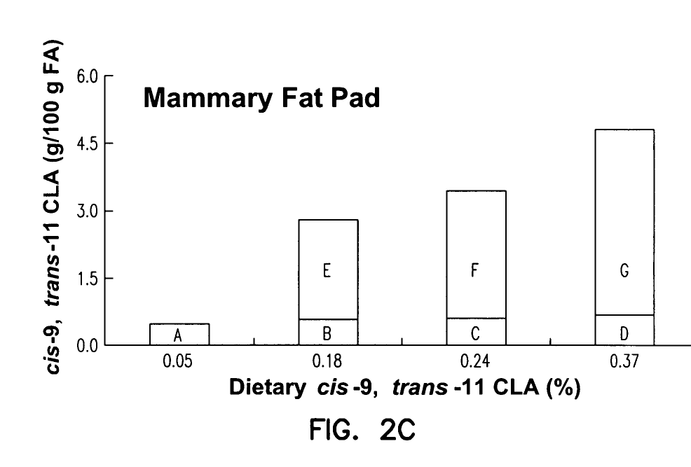

The proportion of most fatty acids was similar for treatments A, B, C, and D. However, tissue and plasma concentrations of vaccenic acid were increased with increasing intake of vaccenic acid (FIG. 1). Likewise, tissue and plasma concentrations of cis-9, trans-11 CLA were increased with increasing dietary intake of cis-9, trans-11 CLA (FIG. 2). Liver concentration of cis-9, trans-11 CLA was increased more than 100% across the range of treatments A to D. However, the lipid content of CLA in both plasma and mammary fat pad was increased by only 38% when treatment A was compared to treatment D. In each tissue, a trend of increasing tissue cis-9, trans-11 CLA was observed with increasing dietary cis-9, trans-11 CLA (FIG. 2; bars corresponding to treatments A, B, C, and D). Liver lipid content of several n-3 fatty acids (Table 4; 20:5, 22:5, 22:6) was also increased, but the basis is unknown.

In contrast, at the same intake level of CLA, a progressive increase in the dietary supply of vaccenic acid (treatments E, F, and G) resulted in even greater cis-9, trans-11 CLA concentrations (FIG. 2). Increasing vaccenic acid from 0.13% in the diet to 0.73% (B vs. E) resulted in 2 to 3 fold increases in tissue and plasma cis-9, trans-11 CLA concentrations. Comparing treatments C and F, increasing dietary vaccenic acid from 0.13% to 1.0% resulted in an approximate three-fold increase in cis-9, trans-11 CLA concentration in both tissues and plasma. Similarly, large increases in cis-9, trans-11 CLA were observed when treatments D and G were compared, corresponding to an increase of vaccenic acid from 0.13% to 1.6%. The two butter sources also differed in concentration of other trans fatty acids (Table 1) and increasing their dietary supply increased the tissue and plasma content of those fatty acids as well (Tables 3, 4, and 5).

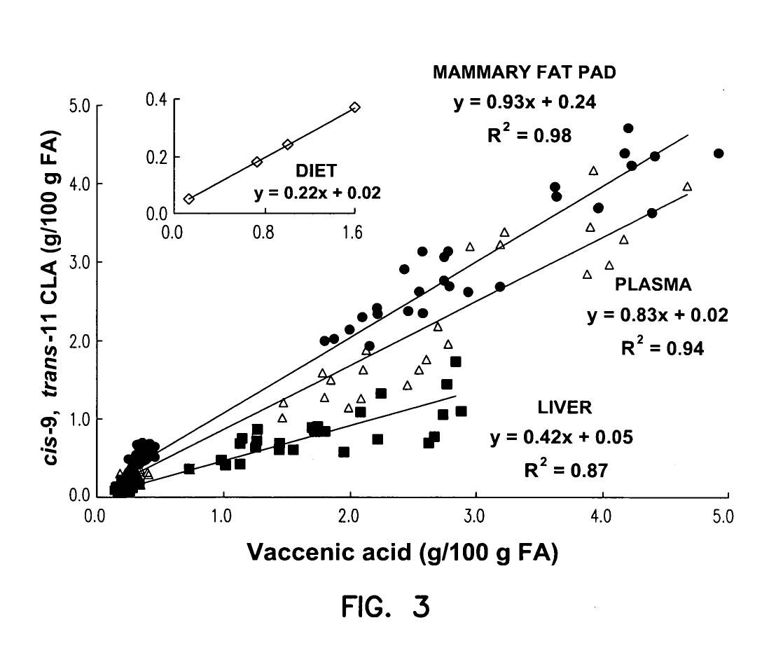

The contribution of dietary vaccenic acid to tissue cis-9, trans-11 CLA is graphically shown in FIG. 3. To provide perspective, the inset in the figure demonstrates the relationship between vaccenic acid and cis-9, trans-11 CLA in the diet. The individual data points for liver, plasma and mammary fat pad from all treatments are shown with their corresponding regression line. Within each tissue, increasing the supply of vaccenic acid linearly increased the content of cis-9, trans-11 CLA. The regression lines show the relative conversion of vaccenic acid to cis-9, trans-11 CLA and the increasing concentration of cis-9, trans-11 CLA when comparing the diet to liver, liver to plasma, and finally the accumulation of cis-9, trans-11 CLA in the mammary fat pad. There was a relative increase in the proportion of vaccenic acid converted to cis-9, trans-11 CLA over the progression from diet to mammary fat pad. The substantial difference in slope between liver and plasma would presumably reflect endogenous synthesis of cis-9, trans-11 CLA by hepatic Δ⁹-desaturase.

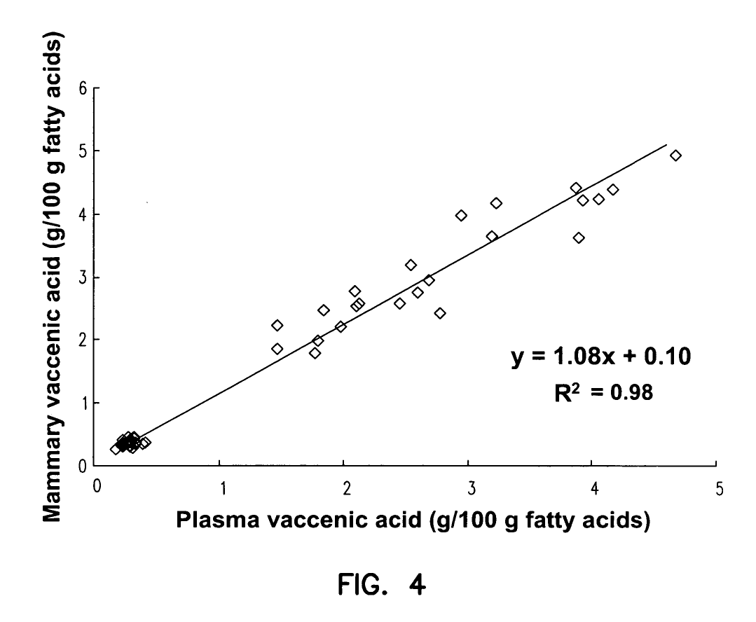

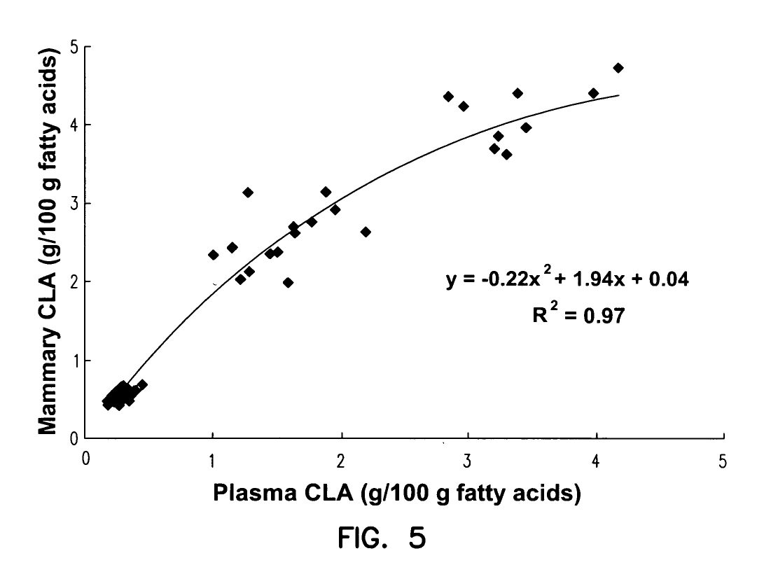

The relative transfer of vaccenic acid from plasma to mammary gland lipids is shown in FIG. 4. This represents a linear relationship. Likewise, the relationship between plasma and mammary content of cis-9, trans-11 CLA is shown in FIG. 5, in this case, a quadratic relationship is observed. The relationship is linear in lower concentrations and appears to approach a plateau at 3% to 4% cis-9, trans-11 CLA in plasma.

### EXAMPLE 2

### The Anticarcinogenic Effect of trans-11 18:1 is Dependent on its Conversion to cis-9, trans-11 CLA by Delta-9 Desaturase

This Example shows that inhibition of delta-9 desaturase, which is the enzyme responsible for conversion of vaccenic acid (VA) to cis-9, trans-11 CLA, negates the inhibitory effect of the high dietary intake of VA on cell proliferation of premalignant lesions.

Materials and Methods

Treatment protocol of animal carcinogenesis experiment. Female Sprague-Dawley rats were purchased from Charles River Breeding Laboratories (Raleigh, N.C.) at 45 d of age; all subsequent procedures were approved by the Roswell Park Cancer Institute Animal Care and Use Committee. Rats were fed the AIN-76 basal diet as described above for 1 wk to acclimate them to the powdered diet. All rats were injected with a single dose of methylnitrosourea (MNU: 50 mg/kg body wt) intraperitoneally at 50 d of age for the induction of premalignant lesions in the mammary gland. Immediately after MNU administration, rats (n=72) were divided into four equal groups, and fed either a low- or high-VA diet, with or without SO (Table 7).

TABLE 7
Design of diets containing control butter fat and/or
VA/CLA-enriched butter fat and sterculic oil (SO)

| | | | | | Fatty Acid | |
|---|---|---|---|---|---|---|
| | Diet Proportion | | | | Content | |
| | | VA/ | Synthetic | | | cis-9, |
| | Control | CLA | cis-9, trans-11 | | trans-11 | trans-11 |
| Dietary | Butter¹ | Butter² | CLA | SO³ | 18:1 | CLA |
| Treatment | Diet % | | | | Diet % | |
| A | 10 | | 0.32 | | 0.13 | 0.37 |
| B | 10 | | 0.32 | 0.40 | 0.13 | 0.37 |
| C | | 10 | | | 1.60 | 0.37 |
| D | | 10 | | 0.40 | 1.60 | 0.37 |

¹Contained 1.30 and 0.51 g/100 g fatty acids as VA and cis-9, trans-11 CLA, respectively.
²Contained 16.28 and 3.76 g/100 g fatty acids as VA and cis-9, trans-11 CLA, respectively.
³Sterculic oil: contained two cyclopropenoic fatty acids; 56% sterculic acid (8-(2-octyl-1-cyclopropenyl) octanoic acid) and 6% malvalic acid (7-(2-octyl-1-cyclopropenyl) heptanoic acid).

As shown in Table 7, the low VA diet included a control butter and the high VA diet utilized a VA/CLA-enriched butter that was produced by feeding cows a natural diet that was specifically formulated for this purpose. Bauman et al. (2000) J. Dairy Sci. 83: 2422-2425. The fatty acid composition of the control and VA-CLA butters as well as the composition of the control and VA/CLA-enriched diets were described previously (Corl, Barbano, et al. 2003 495). To achieve a constant dietary content of cis-9, trans-11 CLA, a synthetic cis-9, trans-11 CLA (Natural, Hovbebygda, Norway; 90% purity) was added to the control butter diets. Rats were fed these diets for 6 wk. Six hr before sacrifice, nine rats from each group were injected i.p. with bromodeoxyuridine (BrdU) at a dose of 50 mg/kg body wt. At necropsy, the whole mammary gland on both sides was excised and processed for histological evaluation of premalignant lesions and BrdU labeling. The remaining nine animals from each group were not injected with BrdU and the liver, plasma and mammary fat pad from these animals were used for fatty acid analysis.

Quantification of premalignant lesions in the mammary gland. The abdominal-inguinal mammary gland chains were fixed in methacarn, and processed in a Tissue-Tek Vacuum Infiltration Processor (Miles Scientific, Elkhart, Ind.). Each mammary gland whole mount was divided into six segments and embedded in paraffin blocks. Ribbons of 5-μm thickness were cut from each block and placed on slides that had been treated with 3-aminopropyltriethoxysilane. Every tenth section was heat-immobilized, deparaffinized in xylene, rehydrated in descending grades of ethanol (100% to 70%), and stained with H&E. These H&E slides were examined under the microscope for the appearance of premalignant lesions using the criteria described by Russo et al. (1982) Differentiation of the mammary gland and susceptibility to carcinogenesis. Breast Cancer Res. Treat. 2:5-73. Once a section showing such pathology was found, the contiguous slides were similarly stained and examined. The size of each premalignant lesion could thus be estimated operationally by the number of serial sections showing the same pathology. Colored micrographs of these premalignant lesions have been published previously by Ip, C., H. J. Thompson, and H. E. Ganther. 2000b. Selenium modulation of cell proliferation and cell cycle biomarkers in normal and premalignant cells of the rat mammary gland. Cancer Epidemiol. Biomarkers Prev. 9:49-54.

Evaluation of proliferative activity by bromodeoxyuridine labeling. Slide preparation for this assay was similar to that described above. Mouse anti-BrdU antibody (Becton Dickinson) was applied at a dilution of 1:40 for 60 min. After the tissue sections were incubated with the primary antibody at room temperature in a humid chamber, they were treated with a biotinylated rabbit secondary antibody against mouse immunoglobulin. This was followed by the addition of streptavidin-horseradish peroxidase, which binds to biotin. Diaminobenzidine was used as the chromogen to generate a brown precipitate due to its reaction with peroxidase. All slides were counterstained with hematoxylin, rinsed, dehydrated, and mounted with Permount. Cells expressing the antigen were identified by a brown stain over the nucleus. Color pictures were taken with a camera mounted on top of the microscope. To avoid bias all hard-copy images were coded so that the person analyzing the data was blinded to the group assignment.

Fatty acid analysis. The −80° C. samples of liver and mammary fat pad were pulverized at liquid nitrogen temperature. Total lipids were extracted from pulverized tissues and plasma using a mixture of hexane and isopropanol by the procedure of Hara & Radin (1978) Anal. Bioch. 90: 420-426. Fatty acids were methylated according to Christie ((1982) J. Lipid Res. 23: 1072-1075) with modifications as described by Corl et al. ((2003) cis-9, trans-i 1 CLA derived endogenously from trans-11 18:1 reduces cancer risk in rats. J. Nutr. 133:2893-2900). Fatty acid methyl esters were analyzed by gas chromatography (Hewlett Packard GC system 6890+with flame ionization detector) using a CP-Sil 88 capillary column (100 m×0.25 mm i.d. with 0.2 μm film thickness; Varian Inc., Walnut Creek, Calif.). A programmed temperature run was used to separate fatty acid methyl esters. The oven temperature was initially maintained at 70° C. for two minutes then increased at 8° C./min to 110° C. and held for four minutes. The temperature was then increased at 5° C./min to 170° C. and held for ten minutes. Finally it was increased at 4° C./min to 225° C. and held for 15 minutes. Injector and detector temperatures were maintained at 250° C. The split ratio was 100:1 and hydrogen was used as the carrier gas at 2.1 mL/min. Fatty acid methyl ester standards were used to identify sample fatty acid methyl esters (Nu-Chek Prep; Elysian, Minn.).

Statistical analyses. The premalignant lesion data were analyzed by the χ² test using a Poisson regression model. McCullagh, P. and J. A. Nelder. 1989. Generalized linear models. Chapman and Hall, London. Differences in BrdU staining between groups were analyzed by using a Kruskal-Wallis rank test. Kruskal, W. H. and W. A. Wallis. 1952. Use of ranks in one criterion variance analysis. J. Am. Stat. Assoc. 47:583-621. Fatty acid data were analyzed by the General Linear Model procedure of SAS (SAS Inc., Cary, N.C.). Analysis of variance (ANOVA) was used to identify the effect of treatment and differences between treatment means were identified using the PDIFF option of the LSMeans command. Treatment effects and differences between means were considered significant when P<0.05.

Results

Modulation of mammary cancer risk and proliferative activity. A high intake of VA reduced the total number of premalignant lesions by almost 50% (Table 8; treatment A vs. C).

TABLE 8
Effect of vaccenic acid (VA), with or without
sterculic oil (SO), on the modulation of mammary
premalignant lesion development

| | Size distribution of premalignant | | | | | Total |
|---|---|---|---|---|---|---|
| | lesions (μm) | | | | | No. of |
| Treatment¹ | <100 | 100-200 | 200-300 | 300-400 | >400 | lesions |
| A | 9 | 15 | 24 | 19 | 16 | 83 |
| D | 10 | 12 | 26 | 17 | 15 | 80 |
| C | 7 | 12 | 11 | 8 | 5 | 43* |
| D | 6 | 16 | 17 | 17 | 12 | 68 |

¹Treatments A and B contained 0.37% VA and treatments C and D contained 1.60% VA. Treatments B and D also contained 0.40% sterculic oil, as detailed in Table 7.
*P < 0.02 for comparison between Group A and C, P < 0.05 for comparison between Group C and D

Premalignant lesions in the mammary gland were categorized into five-size classes and this decrease was accounted for primarily by a reduced population of the larger lesions (>200 μm). Treatment with SO had no effect on the development of lesions in the low VA group (treatment A vs. B), but appeared to reverse the inhibitory effect of the high VA group (treatment C vs. D).

Proliferative activity of premalignant lesions and normal alveolar epithelial cells of the mammary gland was determined by BrdU labeling (Table 9).

TABLE 9
Effect of vaccenic acid (VA), with or without sterculic
oil (SO), on BrdU labeling of premalignant
cells and normal alveolar cells of the mammary gland

% of positively stained cells

| Treatment¹ | Premalignant cells | Alveolar cells |
|---|---|---|
| A | 25.7 ± 2.1 | 14.3 ± 1.6 |
| B | 23.5 ± 2.8 | 15.2 ± 1.5 |
| C | 15.4 ± 1.7* | 13.2 ± 1.5 |
| D | 22.8 ± 3.3 | 13.9 ± 2.2 |

¹Treatments A and B contained 0.37% VA and treatments C and D contained 1.60% VA. Treatments B and D also contained 0.40% sterculic oil, as detailed in Table 7.
*Statistical analysis of BrdU labeling in premalignant cells, P < 0.01 for comparison between Group A and C, P < 0.01 for comparison between Group C and D.

The VA-enriched diet reduced the proportion of BrdU-positive cells in the premalignant lesions (treatment A vs. C), but had no effect on the normal alveolar cell population. Treatment with SO negated the inhibitory effect of the high dietary intake of VA on cell proliferation of the premalignant lesions (treatment C vs. D).

Fatty acid analysis of tissue and plasma lipids. With each tissue and plasma, the fatty acid composition was similar across treatments with the exception of those fatty acids that were either substrates or products of Δ⁹-desaturase (Tables 10, 11, and 12). Increasing the VA content of the diet from 0.13 to 1.60% resulted in substantial increases in the concentration of cis-9, trans-11 CLA. Comparing treatments A and C, the proportion of fatty acids as cis-9, trans-11 CLA was increased by 193, 177 and 123% in liver, plasma, and mammary fat pad, respectively. Treatment with SO did not reduce the fatty acid content of cis-9, trans-11 CLA at the low intake of VA (treatment A vs. B), but at the high intake of VA it resulted in a significant decrease in the concentration of cis-9, trans-11 CLA in the liver, plasma and mammary fat pad. This was greatest in the mammary fat pad where the dietary addition of SO reduced cis-9, trans-11 CLA by almost 40% (treatment C vs. D). The reduced concentration of cis-9, trans-11 CLA in treatment D was associated with an accumulation of VA, consistent with the action of SO inhibiting the endogenous synthesis of cis-9, trans-11 CLA via Δ⁹-desaturase. The presence of SO at both the low and high doses of VA altered additional fatty acid substrate:product pairs associated with Δ⁹-desaturase. This enzyme can add a cis-9 double bond to 14:0, 16:0 and 18:0 to produce cis-9 14:1, cis-9 16:1, cis-9 18:1, respectively, and in most cases, differences in these fatty acid pairs was evident. An exception was when substrate supply was low, at which time the addition of SO to the diet had no effect on these fatty acid pairs; this was apparent at the low dose of VA when SO did not reduce tissue concentrations of cis-9, trans-11 CLA (treatment A vs. B).

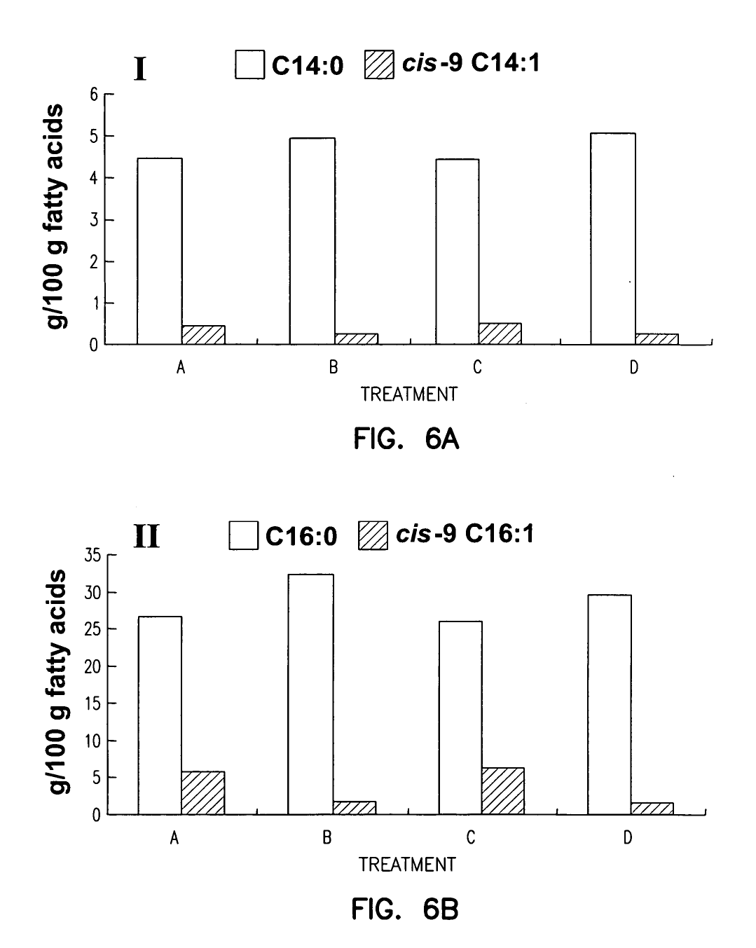

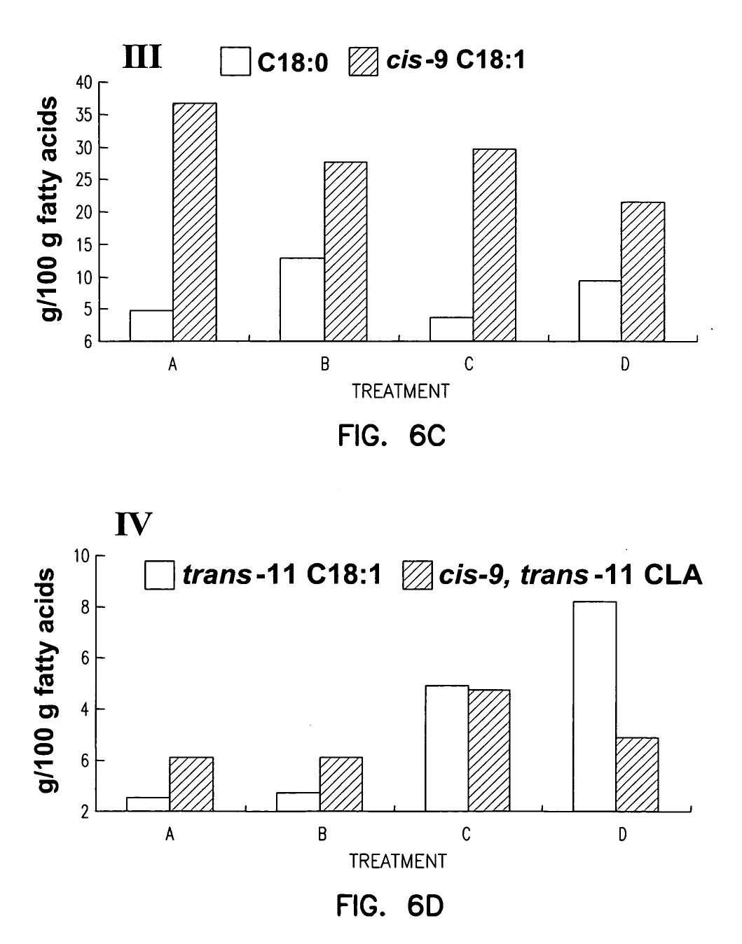

However, comparing the same dietary treatments, SO significantly reduced the mammary fat pad concentrations of cis-9 14:1, cis-9 16:1 and cis-9 18:1, and by the same token, increased the concentrations of 14:0, 16:0 and 18:0 (FIG. 6). Alterations of these fatty acid pairs were similar across the low and high doses of VA (treatments A vs. B and C vs. D) indicating similar efficacy in the inhibition of Δ⁹-desaturase in both treatments (B and D; FIG. 6). Total and individual isomers of trans fatty acids were increased in treatments C and D consistent with their increased supply in the diet.

TABLE 10
Composition of fatty acids from liver lipids of rats fed increasing
amounts of vaccenic acid (VA), with or without sterculic oil (SO)

| | Treatment¹ | | | | | |
|---|---|---|---|---|---|---|
| | A | B | C | D | | |
| Fatty acid | g/100 g fatty acids | | | | se | P² |
| 12:0 | 0.07^{a} | 0.04^{ab} | 0.04^{b} | 0.04^{ab} | 0.01 | <0.05 |
| 14:0 | 0.96^{a} | 0.70^{b} | 0.71^{b} | 0.74^{b} | 0.06 | <0.01 |
| 14:1, cis-9 | 0.11^{a} | 0.06^{b} | 0.09^{a} | 0.06^{b} | 0.01 | <0.001 |
| iso-15:0 | 0.02 | 0.02 | 0.02 | 0.02 | <0.01 | NS |
| antiiso-15:0 | 0.04 | 0.03 | 0.03 | 0.04 | <0.01 | NS |
| 15:0 | 0.22^{a} | 0.17^{b} | 0.21^{a} | 0.21^{a} | 0.01 | <0.01 |
| 16:0 | 19.83^{a} | 19.28^{a} | 17.55^{b} | 18.91^{ab} | 0.50 | <0.05 |
| 16:1, cis-9 | 2.65^{a} | 0.54^{b} | 2.60^{a} | 0.51^{b} | 0.13 | <0.001 |
| 17:0 | 0.35^{bc} | 0.39^{b} | 0.34^{c} | 0.47^{a} | 0.01 | <0.001 |
| 18:0 | 17.26^{c} | 25.67^{a} | 16.08^{c} | 23.81^{b} | 0.48 | <0.001 |
| 18:1, trans-6 to 8 | 0.11^{c} | 0.20^{b} | 0.22^{b} | 0.29^{a} | 0.01 | <0.001 |
| 18:1, trans-9 | 0.16^{c} | 0.10^{d} | 0.37^{a} | 0.21^{b} | 0.01 | <0.001 |
| 18:1, trans-10 | 0.08^{b} | 0.06^{b} | 0.30^{a} | 0.30^{a} | 0.02 | <0.001 |
| 18:1, trans-11 | 0.24^{b} | 0.21^{b} | 2.92^{a} | 3.18^{a} | 0.09 | <0.001 |
| 18:1, trans-12 | 0.39^{b} | 0.44^{b} | 0.68^{a} | 0.69^{a} | 0.02 | <0.001 |
| 18:1, cis-9 | 22.16^{a} | 12.81^{c} | 17.32^{b} | 9.70^{d} | 0.66 | <0.001 |
| 18:2, cis-9, cis-12 | 4.10^{d} | 6.69^{b} | 5.38^{c} | 7.78^{a} | 0.23 | <0.001 |
| CLA, cis-9, trans-11 | 0.84^{c} | 0.77^{c} | 2.46^{a} | 1.35^{b} | 0.09 | <0.001 |
| 18:3, all cis 6, 9, 12 | 0.08^{ab} | 0.09^{a} | 0.09^{a} | 0.06^{b} | 0.01 | <0.05 |
| 18:3, all cis 9, 12, 15 | 0.17^{a} | 0.13^{b} | 0.11^{b} | 0.14^{ab} | 0.01 | <0.01 |
| 20:0 | 0.04 | 0.03 | 0.04 | 0.03 | <0.01 | NS |
| 20:1, cis-11 | 0.02 | 0.03 | 0.03 | 0.03 | 0.01 | NS |
| 20:3, all cis 8, 11, 14 | 0.85^{b} | 1.61^{a} | 0.99^{b} | 1.64^{a} | 0.05 | <0.001 |
| 20:4, all cis 5, 8, 11, 14 | 13.99^{a} | 13.19^{ab} | 12.74^{ab} | 11.02^{b} | 0.58 | <0.01 |
| 20:5, all cis 5, 8, 11, 14, 17 | 0.36^{b} | 0.49^{b} | 0.95^{a} | 0.88^{a} | 0.05 | <0.001 |
| 22:4, all cis 7, 10, 13, 16 | 0.22^{ab} | 0.23^{ab} | 0.17^{b} | 0.24^{a} | 0.02 | <0.05 |
| 22:5, all cis 7, 10, 13, 16, 19 | 0.23^{c} | 0.25^{c} | 0.40^{b} | 0.55^{a} | 0.03 | <0.001 |
| 22:6, all cis 4, 7, 10, 13, 16, 19 | 5.93^{b} | 6.96^{b} | 8.21^{a} | 8.72^{a} | 0.31 | <0.001 |
| Other | 8.51 | 8.81 | 8.92 | 8.38 | 0.20 | NS |

¹Treatments A and B contained 0.37% VA and treatments C and D contained 1.60% VA. Treatments B and D also contained 0.40% sterculic oil, as detailed in Table 7.
²P-value for Single Factor ANOVA comparing treatments A, B, C, and D. When a treatment effect was significant, treatments were compared by t-test and differences indicated by different superscripts (a, b, c, d; P < 0.05).

TABLE 11
Composition of fatty acids from plasma lipids of rats fed increasing
amounts of vaccenic acid (VA), with or without sterculic oil (SO)

| | Treatment¹ | | | | | |
|---|---|---|---|---|---|---|
| | A | B | C | D | | |
| Fatty acid | g/100 g fatty acids | | | | se | P² |
| 12:0 | 0.16^{ab} | 0.20^{a} | 0.07^{b} | 0.15^{ab} | 0.03 | <0.05 |
| 14:0 | 1.35 | 1.41 | 0.88 | 1.29 | 0.18 | NS |
| 14:1, cis-9 | 0.12^{a} | 0.11^{ab} | 0.08^{b} | 0.09^{ab} | 0.01 | <0.05 |
| iso-15:0 | 0.03 | 0.03 | 0.02 | 0.03 | 0.01 | NS |
| antiiso-15:0 | 0.05 | 0.06 | 0.03 | 0.06 | 0.01 | NS |
| 15:0 | 0.30 | 0.27 | 0.26 | 0.30 | 0.02 | NS |
| 16:0 | 20.63^{a} | 22.55^{a} | 18.97^{b} | 21.36^{ab} | 0.78 | <0.01 |
| 16:1, cis-9 | 2.49^{a} | 0.70^{b} | 2.46^{a} | 0.67^{b} | 0.15 | <0.001 |
| 17:0 | 0.26^{c} | 0.37^{a} | 0.27^{c} | 0.43^{a} | 0.24 | <0.001 |
| 18:0 | 13.96^{b} | 20.21^{a} | 12.43^{b} | 18.53^{a} | 0.65 | <0.001 |
| 18:1, trans-6 to 8 | 0.17^{c} | 0.30^{b} | 0.25^{bc} | 0.40^{a} | 0.02 | <0.001 |
| 18:1, trans-9 | 0.25^{c} | 0.21^{c} | 0.41^{a} | 0.35^{b} | 0.02 | <0.001 |
| 18:1, trans-10 | 0.20^{c} | 0.20^{c} | 0.44^{b} | 0.61^{a} | 0.03 | <0.001 |
| 18:1, trans-11 | 0.34^{c} | 0.41^{c} | 3.09^{b} | 4.77^{a} | 0.15 | <0.001 |
| 18:1, trans-12 | 0.38^{d} | 0.55^{c} | 0.76^{b} | 0.99^{a} | 0.05 | <0.001 |
| 18:1, cis-9 | 25.70^{a} | 18.05^{b} | 20.43^{b} | 12.93^{c} | 1.02 | <0.001 |
| 18:2,cis-9, cis-12 | 4.75^{c} | 6.71^{b} | 6.04^{b} | 7.94^{a} | 0.30 | <0.001 |
| CLA, cis-9, trans-11 | 1.13^{c} | 1.30^{c} | 3.13^{a} | 1.89^{b} | 0.09 | <0.001 |
| 18:3, all cis 6, 9, 12 | 0.07^{a} | 0.07^{a} | 0.09^{a} | 0.02^{b} | 0.01 | <0.01 |
| 18:3, all cis 9, 12, 15 | 0.37 | 0.33 | 0.34 | 0.37 | 0.02 | NS |
| 20:0 | 0.49 | 0.42 | 0.57 | 0.43 | 0.08 | NS |
| 20:1, cis-11 | 0.19 | 0.20 | 0.22 | 0.25 | 0.04 | NS |
| 20:3, all cis 8, 11, 14 | 0.64^{c} | 1.26^{ab} | 0.98^{bc} | 1.50^{a} | 0.12 | <0.001 |
| 20:4, all cis 5, 8, 11, 14 | 11.04^{a} | 7.58^{ab} | 9.79^{ab} | 6.35^{b} | 1.01 | <0.01 |
| 20:5, all cis 5, 8, 11, 14, 17 | 0.28^{c} | 0.27^{c} | 0.66^{a} | 0.48^{b} | 0.04 | <0.001 |
| 22:4, all cis 7, 10, 13, 16 | 0.67 | 0.83 | 1.02 | 0.96 | 0.14 | NS |
| 22:5, all cis 7, 10, 13, 16, 19 | 0.10^{b} | 0.11^{b} | 0.25^{a} | 0.26^{a} | 0.03 | <0.001 |
| 22:6, all cis 4, 7, 10, 13, 16, 19 | 2.42^{b} | 2.55^{b} | 3.68^{a} | 3.39^{ab} | 0.27 | <0.01 |
| Other | 11.45 | 12.73 | 12.34 | 13.24 | 0.63 | NS |

¹Treatments A and B contained 0.37% VA and treatments C and D contained 1.60% VA. Treatments B and D also contained 0.40% sterculic oil, as detailed in Table 7.
²P-value for Single Factor ANOVA comparing treatments A, B, C, and D. When a treatment effect was significant, treatments were compared by t-test and differences indicated by different superscripts (a, b, c, d; P < 0.05).

TABLE 12
Composition of fatty acids from mammary fat pad lipids
of rats fed increasing amounts of vaccenic acid (VA),
with or without sterculic oil (SO)

| | Treatment¹ | | | | | |
|---|---|---|---|---|---|---|
| | A | B | C | D | | |
| Fatty acid | g/100 g fatty acids | | | | se | P² |
| 12:0 | 0.94^{ab} | 0.96^{ab} | 0.87^{b} | 1.02^{a} | 0.03 | <0.05 |
| 14:0 | 4.45^{b} | 4.93^{a} | 4.43^{b} | 5.06^{a} | 0.06 | <0.001 |
| 14:1, cis-9 | 0.45^{a} | 0.27^{b} | 0.49^{a} | 0.25^{b} | 0.01 | <0.001 |
| iso-15:0 | 0.06^{c} | 0.07^{c} | 0.08^{b} | 0.09^{a} | <0.01 | <0.001 |
| antiiso-15:0 | 0.11^{bc} | 0.11^{b} | 0.10^{c} | 0.12^{a} | <0.01 | <0.001 |
| 15:0 | 0.46^{b} | 0.47^{b} | 0.47^{b} | 0.54^{a} | 0.01 | <0.001 |
| 16:0 | 26.55^{c} | 32.40^{a} | 25.91^{c} | 29.48^{b} | 0.31 | <0.001 |
| 16:1, cis-9 | 5.91^{a} | 1.70^{b} | 6.29^{a} | 1.61^{b} | 0.12 | <0.001 |
| 17:0 | 0.14^{c} | 0.52^{a} | 0.29^{b} | 0.52^{a} | 0.03 | <0.001 |
| 18:0 | 4.73^{c} | 12.79^{a} | 3.68^{d} | 9.26^{b} | 0.13 | <0.001 |
| 18:1, trans-6 to 8 | 0.13^{d} | 0.19^{c} | 0.28^{b} | 0.41^{a} | 0.01 | <0.001 |
| 18:1, trans-9 | 0.23^{b} | 0.22^{b} | 0.42^{a} | 0.46^{a} | 0.01 | <0.001 |
| 18:1, trans-10 | 0.25^{c} | 0.22^{c} | 0.77^{b} | 0.92^{a} | 0.03 | <0.001 |
| 18:1, trans-11 | 0.54^{c} | 0.74^{c} | 4.89^{b} | 8.20^{a} | 0.08 | <0.001 |
| 18:1, trans-12 | 0.36^{d} | 0.49^{c} | 1.08^{b} | 1.48^{a} | 0.02 | <0.001 |
| 18:1, cis-9 | 36.58^{a} | 27.68^{c} | 29.57^{b} | 21.47^{d} | 0.31 | <0.001 |
| 18:2, cis-9, cis-12 | 7.24 | 6.60 | 6.25 | 6.86 | 0.36 | NS |
| CLA, cis-9, trans-11 | 2.13^{c} | 2.14^{c} | 4.75^{a} | 2.89^{b} | 0.06 | <0.001 |
| 18:3, all cis 6, 9, 12 | 0.04^{a} | 0.02^{b} | 0.02^{b} | 0.02^{b} | <0.01 | <0.001 |
| 18:3, all cis 9, 12, 15 | 0.47^{a} | 0.38^{b} | 0.41^{ab} | 0.41^{ab} | 0.02 | <0.01 |
| 20:0 | 0.08 | 0.09 | 0.09 | 0.09 | <0.01 | NS |
| 20:1, cis-11 | 0.05^{c} | 0.07^{b} | 0.08^{b} | 0.09^{a} | <0.01 | <0.001 |
| 20:3, all cis 8, 11, 14 | 0.03 | 0.02 | 0.03 | 0.03 | <0.01 | NS |
| 20:4, all cis 5, 8, 11, 14 | 0.27^{a} | 0.16^{bc} | 0.20^{b} | 0.14^{c} | 0.01 | <0.001 |
| 20:5, all cis 5, 8, 11, 14, 17 | <0.01 | <0.01 | <0.01 | <0.01 | <0.01 | NS |
| 22:4, all cis 7, 10, 13, 16 | 0.04^{a} | 0.02^{b} | 0.03^{b} | 0.02^{b} | <0.01 | <0.001 |
| 22:5, all cis 7, 10, 13, 16, 19 | 0.01^{b} | <0.01^{c} | 0.03^{a} | 0.02^{ab} | <0.01 | <0.001 |
| 22:6, all cis 4, 7, 10, 13, 16, 19 | 0.04^{a} | 0.02^{b} | 0.05^{a} | 0.04^{a} | 0.01 | <0.001 |
| Other | 7.66^{b} | 7.14^{c} | 8.52^{a} | 8.50^{a} | 0.12 | <0.001 |

¹Treatments A and B contained 0.37% VA and treatments C and D contained 1.60% VA. Treatments B and D also contained 0.40% sterculic oil, as detailed in Table 7.
²P-value for Single Factor ANOVA comparing treatments A, B, C, and D. When a treatment effect was significant, treatments were compared by t-test and differences indicated by different superscripts (a, b, c, d; P < 0.05).

### REFERENCES

Adlof, R. O., Duval, S. & Emken E. A. (2000) Biosynthesis of conjugated linoleic acid in humans. Lipids 35: 131-135.
Andianaivo, A. A., M.-H. Siess, and E. M. Gaydou. 1995. Modifications of hepatic drug metabolizing enzyme activities in rats fed Baobab seed oil containing cyclopropenoid fatty acids. Food Chem. Toxic. 33:377-382.
Aro, A., Männistö, S., Salminen, I., Ovaskainen, M.-L., Kataja, V. & Uusitupa, M. (2000) Inverse association between dietary and serum conjugated linoleic acid and risk of breast cancer in postmenopausal women. Nutr. Cancer 38: 151-157.
Awad, A., Hermann, T., Finsk, C. S. & Horvath, P. J. (1995) 18:1 n7 Fatty acids inhibit growth and decrease inositol phosphatase release in HT-29 cells compared to n9 fatty acids. Cancer Lett. 91: 55-61.
Banni, S., Angioni, E., Casu, V., Melis, M. P., Carta, G., Corongiu, F. P., Thompson, H. & Ip, C. (1999) Decrease in linoleic acid metabolites as a potential mechanism in cancer risk reduction by conjugated linoleic acid. Carcinogenesis 20: 1019-1024.
Banni, S., Angioni, E., Murru, E., Carta, G., Melis, M. P., Bauman, D., Dong, Y. & Ip, C. (2001) Vaccenic acid feeding increases tissue levels of conjugated linoleic acid and suppresses development of premalignant lesions in rat mammary gland. Nutr. Cancer 41: 91-97.
Banni, S., Carta, G., Angioni, E., Murru, E., Scanu, P., Melis, M. P., Bauman, D. E., Fischer, S. M. & Ip, C. (2001) Distribution of conjugated linoleic acid and metabolites in different lipid fractions in the rat liver. J. Lipid Res. 42: 1056-1061.
Bauman, D. E., Barbano, D. M., Dwyer, D. A., & Griinari, J. M. (2000) Technical Note: Production of butter with enhanced conjugated linoleic acid for use in biomedical studies with animal models. J. Dairy Sci. 83: 2422-2425.
Bauman, D. E., L. H. Baumgard, B. A. Corl, and J. M. Griinari. 2001. Conjugated linoleic acid (CLA) and the dairy cow. In: P. C. Gamsworthy and J. Wiseman (Eds.) Recent Advances in Animal Nutrition 2001. pp. 221-250. Nottingham University Press, Nottingham.
Bauman, D. E., B. A. Corl, and D. G. Peterson. 2003. The biology of conjugated linoleic acids in ruminants. In: J.-L. Sebedio, W. W. Christie, and R. O. Adlof (Eds.) Advances in Conjugated Linoleic Acid Research, Volume 2. pp. 146-173. AOCS Press, Champaign, Ill.
Belury, M. A. (2002) Dietary conjugated linoleic acid in health: Physiological effects and mechanisms of action. Ann. Rev. Nutr. 22: 505-531.
Belury, M. (2002) Not all trans-fatty acids are alike: What consumers may lose when we oversimplify nutrition facts. J. Am. Diet. Assoc. 102: 1606.
Cao, J., J.-P. Blond, and J. Bezard. 1993. Inhibition of fatty acid delta-6 and delta-5 desaturase by cyclopropene fatty acids in rat liver microsomes. Biochim. Biophys. Acta 1210:27-34.
Chajés, V., Lavillonniere, F., Ferrari, P., Jourdan, M.-L., Pinault, M., Maillard, V., Sebedio, J.-L. & Bougnoux, P. (2002) Conjugated linoleic acid content in breast adipose tissue is not associated with the relative risk of breast cancer in a population of French patients. Cancer Epidem. Biomarkers Prev. 11: 672-673.
Christie, W. W. (1982) A simple procedure of rapid transmethylation of glycerolipids and cholestryl esters. J. Lipid Res. 23: 1072-1075.
Clément, L., Poirier, H., Niot, I., Bocher, V., Guerre-Millo, M., Krief, S., Staels, B. & Besnard, P. (2002) Dietary trans-10,cis-12 conjugated linoleic acid induces hyperinsulinemia and fatty liver in the mouse. J. Lipid Res. 43: 1400-1409.
Corl, B. A., D. M. Barbano, D. E. Bauman, and C. Ip. 2003. cis-9, trans-11 CLA derived endogenously from trans-11 18:1 reduces cancer risk in rats. J. Nutr. 133:2893-2900.
Corl, B. A., Baumgard, L. H., Dwyer, D. A., Griinari, J. M., Phillips, B. S. and Bauman, D. E. (2001) The role of Δ⁹-desaturase in the production of cis-9, trans-11 CLA. J. Nutr. Bioch. 12: 622-630.
Craig-Schmidt, M. C. (1992) Fatty acid isomers in foods. In: Fatty Acids in Foods and their Health Implications (Chow, C. K., ed.), pp.365-398. Marcel Dekker Inc., New York, N.Y.
Eisele, T. A., P. M. Loveland, D. L. Kruk, T. R. Meyers, R. O. Sinnhuber, and J. E. Nixon. 1982. Effect of cyclopropenoid fatty acids on the hepatic microsomal mixed-function-oxidase system and aflatoxin metabolism in rabbits. Food Chem. Toxic. 20:407-412.
Emken, E. A. (1995) Physicochemical properties, intake, and metabolism. Am. J. Clin. Nutr. 62: 659S-669S.
Gillman, M. W., Cupples, L. A., Gagnon, D., Millen, B. E., Ellison, R. C. & Castelli, W. P. (1997) Margarine intake and subsequent coronary heart disease in men. Epidem. 8: 144-149.
Gläser, K. R., Wenk, C. & Scheeder, M. R. L. (2002) Effects of feeding pigs increasing levels of C 18:1 trans fatty acids on fatty acid composition of backfat and intramuscular fat as well as backfat firmness. Arch. Anim. Nutr. 56: 117-130.
Gomez, F. E., D. E. Bauman, J. M. Ntambi, and B. G. Fox. 2003. Effects of sterculic acid on stearoyl-CoA desaturase in differentiating 3T3-L1 adipocytes. Biochem. Biophys. Res. Commun. 300:316-326.
Griinari, J. M. and D. E. Bauman. 1999. Biosynthesis of conjugated linoleic acid and its incorporation into meat and milk in ruminants. In: M. P. Yurawecz, M. M. Mossoba, J. K. G. Kramer, M. W. Pariza, and G. Nelson (Eds.) Advances in conjugated linoleic acid research. pp. 180-200. AOCS Press.
Griinari, J. M., Corl, B. A., Lacy, S. H., Chouinard, P. Y., Nurmela, K. V. V., & Bauman, D. E. (2000) Conjugated linoleic acid is synthesized endogenously in lactating dairy cows by Δ⁹-desaturase. J. Nutr. 130: 2285-2291.
Hara, A. & Radin, N. S. (1978) Lipid extraction of tissues with a low-toxicity solvent. Anal. Bioch. 90: 420-426.
Harfoot, C. G. and G. P. Hazlewood. 1997. Lipid metabolism in the rumen. In: P. N. Hobson and D. S. Stewart (Eds.) The Rumen Microbial Ecosystem (Second Edition). pp. 382-426. Chapman & Hall, London.
Horvath, P. M. & Ip, C. (1983) Synergistic effect of vitamin E and selenium in chemoprevention of mammary carcinogenesis in rats. Cancer Res. 43: 5335-5341.
Institute of Medicine (2002) Letter report on dietary reference intakes for trans fatty acids.
Ip, C. (1987) Fat and essential fatty acid in mammary carcinogenesis. Am. J. Clin. Nutr. 45: 218-224.
Ip, C., Banni, S., Angioni, E., Carta, G., McGinley, J., Thompson, H. J., Barbano, D. & Bauman, D. (1999) Conjugated linoleic acid-enriched butter fat alters mammary gland morphogenesis and reduces cancer risk in rats. J. Nutr. 129: 2135-2142.
Ip, C., Chin, S. F., Scimeca, J. A. & Pariza, M. W. (1991) Mammary cancer prevention by conjugated dienoic derivative of linoleic acid. Cancer Res. 51: 6118-6124.
Ip, C., Dong, Y., Thompson, H. J., Bauman, D. E. & Ip, M. M. (2001) Control of rat mammary epithelium proliferation by conjugated linoleic acid. Nutr. Cancer 39: 233-238.
Ip, C., Ip, M. M., Loftus, T., Shoemaker, S. & Shea-Eaton, W. (2000) Induction of apoptosis by conjugated linoleic acid in cultured mammary tumor cells and premalignant lesions of the rat mammary gland. Cancer Epidem. Biomarkers Prev. 9: 689-696.
Ip, C., Jiang, C., Thompson, H. J. & Scimeca, J. A. (1997) Retention of conjugated linoleic acid in the mammary gland is associated with tumor inhibition during the post-initiation phase of carcinogenesis. Carcinogenesis 18: 755-759.
Ip, M. M., Masso-Welch, P., Shoemaker, S., Shea-Eaton, W. & Ip, C. (1999) Conjugated linoleic acid inhibits proliferation and induces apoptosis of normal rat mammary epithelial cells in primary culture. Exp. Cell Res. 250: 22-34.
Ip, C., Singh, M., Thompson, H. J. & Scimeca, J. A. (1994) Conjugated linoleic acid suppresses mammary carcinogenesis and proliferative activity of the mammary gland in the rat. Cancer Res. 54: 1212-1215.
Ip, C., H. J. Thompson, and H. E. Ganther. 2000b. Selenium modulation of cell proliferation and cell cycle biomarkers in normal and premalignant cells of the rat mammary gland. Cancer Epidemiol. Biomarkers Prev. 9:49-54.
Jeffcoat, R. and M. R. Pollard. 1977. Studies on the inhibition of the desaturases by cyclopropenoid fatty acids. Lipids 12:480-485.
Kritchevsky, D. 2003. Conjugated linoleic acids in experimental atherosclerosis. In: J.-L. Sebedio, W. W. Christie, and R. O. Adlof (Eds.) Advances in Conjugated Linoleic Acid Research, Volume 2. pp. 293-301. AOCS Press, Champaign, Ill.
Kruskal, W. H. and W. A. Wallis. 1952. Use of ranks in one criterion variance analysis. J. Am. Stat. Assoc. 47:583-621.
Lynch, J. M., A. L. Lock, D. A. Dwyer, R. Norbaksh, D. M. Barbano, and D. E. Bauman. 2004. Flavor and stability of pasteurized milk with elevated levels of conjugated linoleic acid and vaccenic acid. J. Dairy Sci. (in press):(Abstr.).
Masso-Welch, P. A., Zangani, D., Ip, C., Vaughan, M. M., Shoemaker, S., Ramirez, R. A. & Ip, M. M. (2002) Inhibition of angiogenesis by the cancer chemopreventive agent conjugated linoleic acid. Cancer Res. 62: 4383-4389.
McCullagh, P. and J. A. Nelder. 1989. Generalized linear models. Chapman and Hall, London.
Milner, J. A. (1999) Functional foods and health promotion. J. Nutr. 129: 1395S-1397S.
National Research Council. (1996) Carcinogens and Anticarcinogens in the Human Diet. National Academy Press, Washington, D.C.
Nixon, J. E., T. A. Eisele, J. H. Wales, and R. O. Sinnhuber. 1974. Effect of subacute toxic levels of dietary cyclopropenoid fatty acids upon membrane function and fatty acid composition in the rat. Lipids 9:314-321.
Ntambi, J. M. and M. Miyazaki. 2004. Regulation of stearoyl-CoA desaturases and role in metabolism. Prog. Lipid Res. 43:91-104.
Parodi, P. W. 1999. Conjugated linoleic acid and other anticarcinogenic agents of bovine milk fat. J. Dairy Sci. 82:1339-1349.
Parodi, P. W. 2003. Conjugated linoleic acid in food. In: J.-L. Sebedio, W. W. Christie, and R. O. Adlof (Eds.) Advances in Conjugated Linoleic Acid Research, Volume 2. pp. 101-122. AOCS Press, Champaign, Ill. Parodi, P. W. (1997) Cows' milk fat components as potential anticarcinogenic agents. J. Nutr. 127: 1055-1060.
Pietinen, P., Ascherio, A., Korhonen, P., Hartman, A. M., Willett, W. C., Albanes, D. & Virtamo, J. (1997) Intake of fatty acids and risk of coronary heart disease in a cohort of Finnish men. The alpha-tocopherol, beta-carotene cancer prevention study. Am. J. Epidem. 145: 876-887.
Piperova, L. S., Sampugna, J., Teter, B. B., Kalscheur, K. F., Yurawecz, M. P., Ku, Y., Morehouse, K. M., and Erdman, R. A. (2002) Duodenal and milk trans octadecenoic acid and conjugated linoleic acid (CLA) isomers indicate that post-absorptive synthesis is the predominant source of cis-9-containing CLA in lactating dairy cows. J. Nutr. 132: 1235-1241.
Risérus, U., Arner, P., Brismar, K. & Vessby, B. (2002) Treatment with dietary trans10cis12 conjugated linoleic acid causes isomer-specific insulin resistance in obese men with the metabolic syndrome. Diabetes Care 25: 1516-1521.
Risérus, U., Basu, S., Jovinge, S., Fredrikson, G. N., Ärnlöv, J. & Vessby, B. (2002) Supplementation with conjugated linoleic acid causes isomer-dependent oxidative stress and elevated C-reactive protein. A potential link to fatty acid-induced insulin resistance. Circulation 106:1925-1929.
Ritzenthaler, K. L., McGuire, M. K., Falen, R., Shultz, T. D., Dasgupta, N. and McGuire, M. A. (2001) Estimation of conjugated linoleic acid intake by written dietary assessment methodologies underestimates actual intake evaluated by food duplicate methodology. J. Nutr. 131: 1548-1554.
Roche, H. M., Noone, E., Sewter, C., Mc Bennett, S., Savage, D., Gibney, M. J., O'Rahilly, S. & Vidal-Puig, A. J. (2002) Isomer-dependent metabolic effects of conjugated linoleic acid. Insights from molecular markers sterol regulatory element-binding protein-Ic and LXRα Diabetes 51: 2037-2044.
Russo, J., L. K. Tay, and I. H. Russo. 1982. Differentiation of the mammary gland and susceptibility to carcinogenesis. Breast Cancer Res. Treat. 2:5-73.
Salminen, I., Mutanen, M., Jauhiainen, M., & Aro, A. (1998) Dietary trans fatty acids increase conjugated linoleic acid levels in human serum. J. Nutr. Biochem. 9: 93-98.
Santora, J., Palmquist, D. L., & Roehrig, K. L. (2000) Trans-vaccenic acid is desaturated to conjugated linoleic acid in mice. J. Nutr. 130: 208-215.
Stanton, C., J. Murphy, E. McGrath, and R. Devery. 2003. Animal feeding strategies for conjugated linoleic acid enrichment of milk. In: J.-L. Sebedio, W. W. Christie, and R. O. Adlof (Eds.) Advances in Conjugated Linoleic Acid Research, Volume 2. pp. 123-145. AOCS Press, Champaign, Ill.
Toomey, S., H. Roche, D. Fitzgerald, and 0. Belton. 2003. Regression of pre-established atherosclerosis in the apoE(−/−) mouse by conjugated linoleic acid. Biochem. Soc. Trans. 31:1075-1079.
Turpeinen, A. M., Mutanen, M., Aro, A., Salminen, I., Basu, S., Palmquist, D. L. & Griinari, J. M. (2002) Bioconversion of vaccenic acid to conjugated linoleic acid in humans. Am. J. Clin. Nutr. 76: 504-510.
Voorrips, L. E., Brants, H. A. M., Kardinal, A. F. M., Hiddink, G. J., van den Brandt, P. A. & Goldbohn, R. A. (2002) Intake of conjugated linoleic acid, fat, and other fatty acids in relation to postmenopausal breast cancer: the Netherlands Cohort Study on Diet and Caner.
Whigham, L. D., M. E. Cook, and R. L. Atkinson. 2000. Conjugated linoleic acid: Implications for human health. Pharmacol. Res. 42:503-510.
Willet, W. C., Stampfer, M. J., Manson, J. E., Colditz, G. A., Speizer, F. E., Rosner, B., Sampson, L. A. & Hennekens, C. H. (1993) Intake of trans fatty acids and risk of coronary heart disease among wmon. Lancet 341: 581-585.
Williams, C. M. 2000. Dietary fatty acids and human health. Ann. Zootech. 49:165-180.
Wolff, R. L. & Precht, D. (2002) A critique of 50-m Cp-Sil 88 capillary columns used alone to assess trans-unsaturated FA in foods: the case of the TRANSFAIR study. Lipids 37:627-629.
Yurawecz, M. P., Sehat, N., Mossoba, M. M., Roach, J. A. G., Kramer, J. K. G., and Ku, Y. (1999) Variations in isomer distribution in commercially available conjugated linoleic acid. Fett/Lipid 101:277-282.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A composition comprising an effective amount of vaccenic acid and a carrier.

2. The composition of claim 1, wherein the effective amount is sufficient to prevent, treat or decrease tumor formation in a mammal.

3. The composition of claim 1, wherein the effective amount can prevent, treat or decrease tumor formation in mammary tissues.

4. The composition of claim 1, wherein the effective amount is between 0.5 mg and 50 gm.

5. The composition of claim 1, wherein the effective amount is between 250 mg and 5 gm.

6. The composition of claim 1, wherein the composition comprises a triacylglycerol of vaccenic acid.

7. The composition of claim 1, wherein the composition comprises a food product.

8. The composition of claim 7, wherein the food product comprises a diet drink, diet bar, cookie, health bar, salad dressing, sauce, cream, whipped cream, ice cream, butter, milk shake, health drink, powdered drink mix, powdered cake mix, powdered snack bar mix, margarine, cooking oil, lard, prepared frozen meal, candy, cracker, snack chip, meat product, milk, cheese, or yogurt.

9. The composition of claim 1, wherein the composition comprises a health supplement.

10. The composition of claim 9, wherein the health supplement comprises a tablet or capsule.

11. A method for preventing or treating cancer in a mammal comprising administering vaccenic acid to the mammal.

12. The method of claim 11, wherein the vaccenic acid is present in a therapeutically effective amount.

13. The method of claim 12, wherein the therapeutically effective amount is sufficient to increase tissue accumulation of conjugated linoleic acid in a mammal.

14. The method of claim 12, wherein the therapeutically effective amount is between 0.5 mg and 50 gm.

15. The method of claim 12, wherein the therapeutically effective amount is between 250 mg and 5 gm.

16. The method of claim 11, wherein the vaccenic acid comprises a triacylglycerol of vaccenic acid.

17. The method of claim 11, wherein the vaccenic acid is formulated into a food product.

18. The method of claim 17, wherein the food product comprises a diet drink, diet bar, cookie, health bar, salad dressing, sauce, cream, whipped cream, ice cream, butter, milk shake, health drink, powdered drink mix, powdered cake mix, powdered snack bar mix, margarine, cooking oil, lard, prepared frozen meal, candy, cracker, snack chip, meat product, milk, cheese, or yogurt.

19. The method of claim 11, wherein the vaccenic acid is formulated into a health supplement.

20. The method of claim 19, wherein the health supplement comprises a tablet or capsule.

21. The method of claim 11, wherein the cancer is melanoma, non-small cell lung cancer, small cell lung cancer, renal cancer, colorectal cancer, breast cancer, pancreatic cancer, gastric cancer, bladder cancer, ovarian cancer, uterine cancer, lymphoma, prostate carcinoma, adenocarcinoma, ovarian adenocarcinoma, renal adenocarcinoma, mammary adenocarcinoma, lung adenocarcinoma, pancreatic adenocarcinoma or non-seminomal testis carcinoma tissues.

22. The method of claim 11, wherein the cancer is breast cancer.

23. A method for increasing tissue accumulation of conjugated linoleic acid in a mammal comprising administering vaccenic acid to the mammal.

24. The method of claim 23, wherein the vaccenic acid is present in a therapeutically effective amount.

25. The method of claim 24, wherein the therapeutically effective amount is sufficient to increase tissue accumulation of conjugated linoleic acid in a mammal.

26. The method of claim 24, wherein the therapeutically effective amount is between 0.5 mg and 50 gm.

27. The method of claim 24, wherein the therapeutically effective amount is between 250 mg and 5 gm.

28. The method of claim 23, wherein the vaccenic acid comprises a triacylglycerol of vaccenic acid.

29. The method of claim 23, wherein the vaccenic acid is formulated into a food product.

30. The method of claim 29, wherein the food product comprises a diet drink, diet bar, cookie, health bar, salad dressing, sauce, cream, whipped cream, ice cream, butter, milk shake, health drink, powdered drink mix, powdered cake mix, powdered snack bar mix, margarine, cooking oil, lard, prepared frozen meal, candy, cracker, snack chip, meat product, milk, cheese, or yogurt.

31. The method of claim 23, wherein the vaccenic acid is formulated into a health supplement.

32. The method of claim 31, wherein the health supplement comprises a tablet or capsule.
